(19) United States
(12) Patent Application Publication
(10) Pub. No.: US 2020/0354452 A1
(43) Pub. Date: Nov. 12, 2020

(54) CARS AND BISPECIFIC ANTIBODIES FOR TREATMENT OF MANTLE CELL LYMPHOMA

(71) Applicant: City of Hope, Duarte, CA (US)

(72) Inventors: Hong Qin, Upland, CA (US); Larry Kwak, Pasadena, CA (US); Guowei Wei, San Dimas, CA (US)

(21) Appl. No.: 16/651,959

(22) PCT Filed: May 4, 2018
(86) PCT No.: PCT/US2018/031232

§ 371 Date: Mar. 27, 2020

Related U.S. Application Data
(60) Provisional application No. 62/566,020, filed on Sep. 29, 2017.

(51) Int. Cl. C07K 16/28; A61P 35/00; C07K 16/30

(52) U.S. Cl. CPC C07K 16/2809; A61P 35/00; C07K 16/3061; C07K 2317/21; C07K 2317/31; A61K 2039/505; C07K 2317/73; C07K 2319/03; C07K 2319/33; C07K 2317/24; C07K 2317/56

(57) ABSTRACT

Provided herein are, inter alia, compositions and methods for the treatment of cancer. The compositions include, for example, a chimeric antigen receptor or a bispecific antibody comprising a free immunoglobulin light chain VL, inter alia, useful for targeting and killing human mantel cell lymphoma (MCL) cells. Due to their ability to differentially bind MCL cells versus non-cancer cells, the compositions provided herein may be used, for example, for therapeutic and diagnostic purposes.

### CROSS REFERENCE TO RELATED APPLICATIONS

The present application is the 35 U.S.C. § 371 National Stage Application of International Application No. PCT/US2018/031232, filed on May 4, 2018, which claims priority to U.S. Provisional Application No. 62/566,020, filed on Sep. 29, 2017. The entire contents of the foregoing are incorporated herein by reference.

### SEQUENCE LISTING

The instant application contains a Sequence Listing which has been submitted electronically in ASCII format and is hereby incorporated by reference in its entirety. Said ASCII copy, created on May 11, 2020, is named SequenceListing.txt and is 37000 bytes in size.

### BACKGROUND

Immunotherapeutics are currently developed against a range of targets and employ unique mechanisms of action to combat tumors. However, all pursue cell surface proteins that are simultaneously present on malignant cells and their benign counterparts. Consequently, current antibody-based antitumor therapeutics will inevitably deplete healthy, non-malignant cells in the process of eliminating tumors. Chimeric antigen receptor T-cell (CART) therapies are a prime example where ubiquitous, tumor associated antigens are targeted causing adverse effects such as lymphocytopenia. Equally important to specify is the origin of the parent antibody. Many immunotherapeutics are derived from murine generated antibodies, which could elicit immune responses against these components and potentially abrogate the efficacy of the therapy. Thus, there is a need in the art for immunotherapeutics that can distinguish and selectively kill cancer cells while leaving healthy cells unharmed. Provided herein are recombinant protein compositions and methods of using the same addressing these and other problems in the art.

### BRIEF SUMMARY OF THE INVENTION

In an aspect is provided a recombinant protein including: (i) an antibody region including a light chain variable domain including a CDR L1 as set forth in SEQ ID NO:1, a CDR L2 as set forth in SEQ ID NO:2, and a CDR L3 as set forth in SEQ ID NO:3; and (ii) a transmembrane domain.

In an aspect is provided a recombinant protein including: (i) a first antibody region capable of binding an effector cell ligand; and (ii) a second antibody region, including a light chain variable domain including a CDR L1 as set forth in SEQ ID NO:1, a CDR L2 as set forth in SEQ ID NO:2 and a CDR L3 as set forth in SEQ ID NO:3. In embodiments, the light chain variable domain includes the sequence of SEQ ID NO:4.

In an aspect is provided an isolated nucleic acid encoding a recombinant protein as provided herein including embodiments thereof.

In an aspect is provided a pharmaceutical composition including a therapeutically effective amount of a recombinant protein as provided herein, including embodiments thereof, and a pharmaceutically acceptable excipient.

In an aspect is provided a method of treating cancer in a subject in need thereof, the method including administering to a subject a therapeutically effective amount of a recombinant protein as provided herein, including embodiments thereof, thereby treating cancer in the subject.

In an aspect is provided a method of inhibiting proliferation of a cell, the method including: (i) contacting a cell with a recombinant protein as provided herein, including embodiments thereof, thereby forming a contacted cell; and (ii) allowing the recombinant protein as provided herein, including embodiments thereof, to bind to the contacted cell, thereby inhibiting proliferation of the cell.

Described herein are methods for using targeted CAR T cells to treat mantle cell lymphoma. The methods entail the use of CAR T cells that express a CAR that includes include a light chain variable domain includes the sequence of SEQ ID NO:4 or a light chain variable region (e.g., human) comprising a CDR L1 as set forth in SEQ ID NO:1, a CDR L2 as set forth in SEQ ID NO:2, and a CDR L3 as set forth in SEQ ID NO:3 or a light chain variable region that includes a variant of SEQ ID NO:4 having no more than 5 single amino acid substitutions provided that the substitutions are not within the CDR L1 as set forth in SEQ ID NO:1, CDR L2 as set forth in SEQ ID NO:2, and CDR L3 as set forth in SEQ ID NO:3. The CAR also includes, a spacer, a transmembrane domain, a co-stimulatory domain, and a CD3 ζ signaling domain.

In various embodiments, the CAR comprises: a transmembrane domain selected from: a CD4 transmembrane domain or variant thereof having 1-5 amino acid modifications, a CD8 transmembrane domain or variant thereof having 1-5 amino acid modifications, a CD28 transmembrane domain or a variant thereof having 1-5 amino acid modifications; a costimulatory domain selected from: a 4-IBB costimulatory domain or a variant thereof having 1-5 amino acid modifications and an CD28 costimulatory domain or a variant thereof having 1-5 amino acid modifications; a CD3 ζ signaling domain of a variant thereof having 1-5 amino acid modifications; and a spacer region having 20-150 amino acids located between the light chain variable domain and the transmembrane domain.

In various embodiments: the costimulatory domain is selected from the group consisting of: a 4-IBB costimulatory domain and variants thereof having 1-5 amino acid modifications; the transmembrane domain is a CD4 transmembrane domain or variant thereof having 1-5 amino acid modifications; the transmembrane domain is a CD4 transmembrane domain; the CAR comprises two different costimulatory domains selected from the group consisting of a CD28 costimulatory domain or a variant thereof having 1-5 amino acid modifications, a 4-IBB costimulatory domain or a variant thereof having 1-5 amino acid modifications and an OX40 costimulatory domain or a variant thereof having 1-5 amino acid modifications; the chimeric antigen receptor comprises an antibody light chain variable region having the amino acid sequence of SEQ ID NO: 4 or a variant thereof having 1-2 amino acid modifications; a transmembrane domain selected from: a CD4 transmembrane domain or variant thereof having 1-2 amino acid modifications, a CD8 transmembrane domain or variant thereof having 1-2 amino acid modifications, a CD28 transmembrane domain or a variant thereof having 1-2 amino acid modifications, and a CD3 transmembrane domain or a variant thereof having 1-2 amino acid modifications; a 4-IBB costimulatory domain; or a variant thereof having 1-2 amino acid modifications; and CD3 ζ signaling domain of a variant thereof having 1-2 amino acid modifications; and a spacer region having 20-150 amino acids located between the scFv and the transmembrane domain; the spacer region comprises an amino acid sequence selected from the group consisting of SEQ ID NOs: ______ or a variant thereof having 1-5 amino acid modifications; the spacer comprises an IgG hinge region; the spacer comprises 10-50 amino acids; the 4-1BB costimulatory domain comprises the amino acid sequence of SEQ ID NO: 24 or a variant thereof having 1-5 amino acid modifications; the CD3ζ signaling domain comprises the amino acid sequence of SEQ ID NO:21; a linker of 3 to 15 amino acids is located between the costimulatory domain and the CD3 ζ signaling domain or variant thereof; and the CAR comprises the amino acid sequence of SEQ ID NO: ______ or a variant thereof having 1-5 amino acid modifications.

Also described here is an expression vector and a viral vector comprising the nucleic acid molecules described herein. Also described is a population of human T cells transduced by a vector comprising a nucleic acid molecule described herein. Also described is a method of treating cancer in a patient comprising administering a population of autologous or allogeneic human T cells transduced by a vector comprising a nucleic acid molecule described herein. In embodiments of the method: the population of human T cells comprise central memory T cells.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

A “polycistronic RNA” as provided herein refers to an RNA sequence including more than one (e.g., 2, 3, 4, 5, 6, 7) open reading frame (nucleic acid sequence encoding a polypeptide). A polycistronic RNA may include one promoter controlling the expression of all open reading frames encoded by the polycistronic RNA. In embodiments, the polycistronic RNA includes more than one promoter and one or more of the open reading frames included in the polycistronic RNA are expressed by an independent promoter.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection, and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refer to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., an antibody construct or antigen binding domain provided herein) in other proteins with different numbering systems. For example, by performing a simple sequence alignment with a protein (e.g., an antibody construct or antigen binding domain provided herein) the identity and location of residues corresponding to specific positions of said protein are identified in other protein sequences aligning to said protein. For example, a selected residue in a selected antibody construct (or antigen binding domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or antigen binding domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “peptidyl”, “peptidyl moiety” or “peptide moiety” refers to a monovalent peptide and may be alternatively referred to as a portion of a peptide or a portion of a polypeptide.

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified, e.g., labeled.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of anyone of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

A “label,” “detectable agent,” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable light chain provided herein includes in N-terminal to C-terminal direction a CDR L1, a CDR L2 and a CDR L3.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

A “therapeutic agent” as referred to herein, is a composition useful in treating or preventing a disease such as cancer.

The phrase “specifically (or selectively) binds to an antibody” or “specifically (or selectively) immunoreactive with,” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions typically requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or antibody, antibody variant, antibody region or fragment thereof.

A “CD3 protein” as referred to herein includes any of the recombinant or naturally-occurring forms of the Cluster of Differentiation 3 (CD3) proteins or variants or homologs thereof that comprise the CD3 complex that mediates signal transduction and maintains CD3 complex activity (e.g. within at least 50%, 80%, 90%, 95%, 96%, 97%, 98%, 99% or 100% activity compared to the CD3 complex). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3 proteins in the CD3 complex.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting” may include allowing two species to react, interact, or physically touch (e.g., bind), wherein the two species may be, for example, an antibody construct as described herein and a cancer protein. In embodiments, contacting includes, for example, allowing an antibody construct to bind to a cancer protein expressed on a cancer cell.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In some embodiments, the sample is obtained from a human.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma (Mantel cell lymphoma), head and neck cancer, colorectal cancer, prostate cancer, pancreatic cancer, melanoma, breast cancer, neuroblastoma).

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P388 leukemia model is widely accepted as being predictive of in vivo anti-leukemic activity. It is believed that a compound that tests positive in the P388 assay will generally exhibit some level of anti-leukemic activity in vivo regardless of the type of leukemia being treated. Accordingly, the present application includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., cancer (e.g. leukemia, lymphoma, B cell lymphoma, or multiple myeloma)) means that the disease (e.g. cancer, (e.g. leukemia, lymphoma, B cell lymphoma, or multiple myeloma)) is caused by (in whole or in part), or a symptom of the disease is caused by (in whole or in part) the substance or substance activity or function.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. For example, certain methods herein treat cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia lymphoblastic leukemia, chronic lymphocytic leukemia, hairy cell leukemia cancer cell), lymphoma (e.g., mantle cell lymphoma (MCL), follicular lymphoma, diffuse large B-cell lymphoma, marginal zone lymphoma, Burkitt's lymphoma), head and neck cancer, colorectal cancer, prostate cancer, pancreatic cancer, melanoma, breast cancer, neuroblastoma). For example certain methods herein treat cancer by decreasing or reducing or preventing the occurrence, growth, metastasis, or progression of cancer; or treat cancer by decreasing a symptom of cancer. Symptoms of cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) would be known or may be determined by a person of ordinary skill in the art.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

Provided herein are, inter alia, recombinant proteins (e.g., a chimeric antigen receptor, a bispecific antibody) for the treatment of cancer. The chimeric antigen receptor and bispecific antibody provided herein include a light chain variable domain capable of binding to human mantel cell lymphoma (MCL) cells. The recombinant proteins provided herein are, inter alia, useful for targeting and killing cancer cells while leaving healthy cells unharmed. The recombinant proteins provided herein, including embodiments thereof, are capable of specifically binding to human mantel cell lymphomas (MCLs) and causing targeted lysis of MCL cells in the presence of effector cells (e.g., NK cells). Surprisingly, the recombinant proteins provided herein do not bind to non-cancer (healthy) cells, thereby preventing adverse effects otherwise caused by unspecific killing of healthy cells. Due to their ability to differentially bind cancer cells versus non-cancer cells, the recombinant proteins provided herein are highly efficient and efficacious agents which may be used for therapeutic and diagnostic purposes. In embodiments, the recombinant protein is a chimeric antigen receptor (CAR). In embodiments, the recombinant protein is a bispecific antibody.

In an aspect is provided a recombinant protein including: (i) an antibody region including a light chain variable domain including a CDR L1 as set forth in SEQ ID NO:1, a CDR L2 as set forth in SEQ ID NO:2, and a CDR L3 as set forth in SEQ ID NO:3; and (ii) a transmembrane domain.

An “antibody region” as provided herein refers to a monovalent or multivalent protein moiety that forms part of the protein provided herein including embodiments thereof and that is capable of binding an antigen (epitope). The antibody region provided herein may include a domain of an antibody or fragment (e.g., Fab) thereof. Thus, the antibody region may include a light chain variable domain (VL) and/or a heavy chain variable domain (VH). In embodiments, the antibody region provided herein includes a light chain variable (VL) domain. In embodiments, the antibody region is a light chain variable (VL) domain.

As described above, a “light chain variable (VL) domain” as provided herein refers to a peptide (e.g., peptide domain) or peptidyl moiety capable of binding an antigen. A light chain variable (VL) domain as provided includes CDR sequences and framework region (FR) sequences of the light chain of an antibody, an antibody variant or fragment thereof. In embodiments, the light chain variable (VL) domain includes CDR L1 (SEQ ID NO:1), CDR L2 (SEQ ID NO:2), and CDR L3 (SEQ ID NO:3) of an antibody light chain. In embodiments, the light chain variable domain includes the sequence of SEQ ID NO4 In embodiments, the light chain variable domain is the sequence of SEQ ID NO:4.

A “transmembrane domain” as provided herein refers to a polypeptide forming part of a biological membrane. The transmembrane domain provided herein is capable of spanning a biological membrane (e.g., a cellular membrane) from one side of the membrane through to the other side of the membrane. In embodiments, the transmembrane domain spans from the intracellular side to the extracellular side of a cellular membrane. Transmembrane domains may include non-polar, hydrophobic residues, which anchor the proteins provided herein including embodiments thereof in a biological membrane (e.g., cellular membrane of a T cell). Any transmembrane domain capable of anchoring the proteins provided herein including embodiments thereof are contemplated. Non-limiting examples of transmembrane domains include the transmembrane domains of CD28, CD8, CD4, CD3ζ, or CD8α. In embodiments, the transmembrane domain is a CD8α transmembrane domain, a CD28 transmembrane domain, a CD4 transmembrane domain, or a CD3 transmembrane domain. In embodiments, the transmembrane domain is a CD28 transmembrane domain. In embodiments, the transmembrane domain is a CD8 transmembrane domain. In embodiments, the transmembrane domain is a CD4 transmembrane domain. In embodiments, the transmembrane domain is a CD3 transmembrane domain. In embodiments, the transmembrane domain is a CD8α transmembrane domain. In embodiments, the CD8α transmembrane domain includes an amino acid sequence encoded by the sequence of SEQ ID NO:6. In embodiments, the CD8α transmembrane domain is an amino acid sequence encoded by the sequence of SEQ ID NO:6.

In embodiments, the recombinant protein as provided herein, including embodiments thereof, further includes an intracellular co-stimulatory signaling domain. An “intracellular co-stimulatory signaling domain” as provided herein includes amino acid sequences capable of providing co-stimulatory signaling in response to binding of an antigen to the antibody region provided herein including embodiments thereof. In embodiments, the signaling of the co-stimulatory signaling domain results in production of cytokines and proliferation of the T cell expressing the same. In embodiments, the intracellular co-stimulatory signaling domain is a CD28 intracellular co-stimulatory signaling domain, a 4-1BB intracellular co-stimulatory signaling domain, a ICOS intracellular co-stimulatory signaling domain, or an OX-40 intracellular co-stimulatory signaling domain. In embodiments, the intracellular co-stimulatory signaling domain is a CD28 intracellular co-stimulatory signaling domain. In embodiments, the intracellular co-stimulatory signaling domain is a ICOS intracellular co-stimulatory signaling domain. In embodiments, the intracellular co-stimulatory signaling domain is an OX-40 intracellular co-stimulatory signaling domain. In embodiments, the intracellular co-stimulatory signaling domain is a 4-1BB intracellular co-stimulatory signaling domain. In embodiments, the 4-1BB intracellular co-stimulatory signaling domain includes an amino acid sequence encoded by the sequence of SEQ ID NO: ______. In some embodiments, the 4-1BB intracellular co-stimulatory signaling domain is an amino acid sequence encoded by the sequence of SEQ ID NO: ______.

In embodiments, the recombinant protein as provided herein including embodiments thereof, further includes an intracellular T-cell signaling domain. An “intracellular T-cell signaling domain” as provided herein includes amino acid sequences capable of providing primary signaling in response to binding of an antigen to the antibody region provided herein including embodiments thereof. In embodiments, the signaling of the intracellular T-cell signaling domain results in activation of the T cell expressing the same. In embodiments, the signaling of the intracellular T-cell signaling domain results in proliferation (cell division) of the T cell expressing the same. In embodiments, the signaling of the intracellular T-cell signaling domain results in expression by said T cell of proteins known in the art to be characteristic of activated T cells (e.g., CTLA-4, PD-1, CD28, CD69). In embodiments, the intracellular T-cell signaling domain includes the signaling domain of the zeta chain of the human CD3 complex. In embodiments, the intracellular T-cell signaling domain is a CD3ζ intracellular T-cell signaling domain. In embodiments, the CD3ζ intracellular T-cell signaling domain includes an amino acid sequence encoded by the sequence of SEQ ID NO: ______. In embodiments, the CD3ζ intracellular T-cell signaling domain is an amino acid sequence encoded by the sequence of SEQ ID NO: ______.

The domains described above (e.g., light chain variable domain, transmembrane domain, intracellular co-stimulatory domain, intracellular T-cell signaling domain) can have a specific order in the recombinant protein from the N-terminus to the C-terminus. Thus, in embodiments, from the N-terminus to the C-terminus, the recombinant protein includes a light chain variable domain as provided herein including embodiments thereof, a transmembrane domain as provided herein including embodiments thereof, an intracellular co-stimulatory domain as provided herein including embodiments thereof, and a intracellular T-cell signaling domain as provided herein including embodiments thereof. In one embodiment, from the N-terminus to the C-terminus, the recombinant protein includes a light chain variable domain of SEQ ID NO:4, a CD8α transmembrane domain encoded by SEQ ID NO: ______, a 4-1BB intracellular co-stimulatory domain encoded by SEQ ID NO: ______, and a CD3ζ intracellular T-cell signaling domain encoded by SEQ ID NO: ______.

In embodiments, the recombinant protein provided herein, including embodiments thereof, further includes a detectable domain. A “detectable domain” as provided herein is a peptide moiety detectable by spectroscopic, photochemical, biochemical, immunochemical, chemical, or other physical means. For example, a detectable domain as provided herein may be a protein or other entity which can be made detectable, e.g., by incorporating a radiolabel or being reactive to an antibody specifically. Any appropriate method known in the art for conjugating an antibody to the label may be employed, e.g., using methods described in Hermanson, Bioconjugate Techniques 1996, Academic Press, Inc., San Diego. In the present invention, a detectable domain is used to confirm transfection of T cells.

In embodiments, detectable domain is a fluorescent protein. In embodiments, the detectable domain is EGFP. In embodiments, detectable domain is a truncated EGFR (EGFRt) domain. The term “EGFRt” refers to a truncated epidermal growth factor receptor protein lacking intracellular signaling capabilities. The EGFRt contains only the transmembrane domain, Domain III, and Domain IV of wild-type human EGFR.

As used herein, EGFRt is an inert cell surface molecule which functions as a detectable domain to identify T cells transduced with a nucleic acid encoding the CAR polypeptide provided herein or individual domains thereof (e.g., light chain variable domain, transmembrane domain, intracellular co-stimulatory domain, intracellular T-cell signaling domain). The detectable domain (e.g., EGFRt) may form part of the recombinant protein provided herein (CAR polypeptide) or it may be present as a separate protein, not forming part of the CAR polypeptide. Thus, the detectable domain (e.g., EGFRt) may be encoded by a nucleic acid sequence that forms part of the same nucleic acid encoding the CAR polypeptide provided herein or individual domains thereof (e.g., light chain variable domain, transmembrane domain, intracellular co-stimulatory domain, intracellular T-cell signaling domain) or the detectable domain (e.g., EGFRt) may be encoded by a separate nucleic acid sequence. Where the detectable domain (e.g., EGFRt) is encoded by a nucleic acid sequence forming part or the same nucleic acid encoding the CAR polypeptide domains, it may be While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, N Y 1989). Any methods, devices and materials similar or equivalent to those described herein can be used in the practice of this invention. The following definitions are provided to facilitate understanding of certain terms used frequently herein and are not meant to limit the scope of the present disclosure.

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

The term “gene” means the segment of DNA involved in producing a protein; it includes regions preceding and following the coding region (leader and trailer) as well as intervening sequences (introns) between individual coding segments (exons). The leader, the trailer, as well as the introns, include regulatory elements that are necessary during the transcription and the translation of a gene. Further, a “protein gene product” is a protein expressed from a particular gene.

A “polycistronic RNA” as provided herein refers to an RNA sequence including more than one (e.g., 2, 3, 4, 5, 6, 7) open reading frame (nucleic acid sequence encoding a polypeptide). A polycistronic RNA may include one promoter controlling the expression of all open reading frames encoded by the polycistronic RNA. In embodiments, the polycistronic RNA includes more than one promoter and one or more of the open reading frames included in the polycistronic RNA are expressed by an independent promoter.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection, and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g, Ford et al. (2001) Gene Therapy 8:1-4 and Prochiantz (2007) Nat. Methods 4:119-20.

The term “plasmid” or “expression vector” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that may be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refer to the numbering of the residues of a specified reference sequence when the given amino acid or polynucleotide sequence is compared to the reference sequence. An amino acid residue in a protein “corresponds” to a given residue when it occupies the same essential structural position within the protein as the given residue. One skilled in the art will immediately recognize the identity and location of residues corresponding to a specific position in a protein (e.g., an antibody construct or antigen binding domain provided herein) in other proteins with different numbering systems. For example, by performing a simple sequence alignment with a protein (e.g., an antibody construct or antigen binding domain provided herein) the identity and location of residues corresponding to specific positions of said protein are identified in other protein sequences aligning to said protein. For example, a selected residue in a selected antibody construct (or antigen binding domain) corresponds to light chain threonine at Kabat position 40, when the selected residue occupies the same essential spatial or other structural relationship as a light chain threonine at Kabat position 40. In some embodiments, where a selected protein is aligned for maximum homology with the light chain of an antibody (or antigen binding domain), the position in the aligned selected protein aligning with threonine 40 is said to correspond to threonine 40. Instead of a primary sequence alignment, a three dimensional structural alignment can also be used, e.g., where the structure of the selected protein is aligned for maximum correspondence with the light chain threonine at Kabat position 40, and the overall structures compared. In this case, an amino acid that occupies the same essential position as threonine 40 in the structural model is said to correspond to the threonine 40 residue.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids sequences encode any given amino acid residue. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

The term “peptidyl”, “peptidyl moiety” or “peptide moiety” refers to a monovalent peptide and may be alternatively referred to as a portion of a peptide or a portion of a polypeptide.

The term “recombinant” when used with reference, for example, to a cell, a nucleic acid, a protein, or a vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified, e.g., labeled.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A protein that is the predominant species present in a preparation is substantially purified.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, optionally 65%, 70%, 75%, 80%, 85%, 90%, 95%, 98%, or 99% identity over a specified region, e.g., of the entire polypeptide sequences of the invention or individual domains of the polypeptides of the invention), when compared and aligned for maximum correspondence over a comparison window, or designated region as measured using one of the following sequence comparison algorithms or by manual alignment and visual inspection. Such sequences are then said to be “substantially identical.” This definition also refers to the complement of a test sequence. Optionally, the identity exists over a region that is at least about 50 nucleotides in length, or more preferably over a region that is 100 to 500 or 1000 or more nucleotides in length.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of, e.g., a full length sequence or from 20 to 600, about 50 to about 200, or about 100 to about 150 amino acids or nucleotides in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross-reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

A “label,” “detectable agent,” or “detectable moiety” is a composition detectable by appropriate means such as spectroscopic, photochemical, biochemical, immunochemical, chemical, magnetic resonance imaging, or other physical means. For example, useful detectable agents include ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y. ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable moiety is a monovalent detectable agent or a detectable agent capable of forming a bond with another composition.

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An “antibody variant” as provided herein refers to a polypeptide capable of binding to an antigen and including one or more structural domains (e.g., light chain variable domain, heavy chain variable domain) of an antibody or fragment thereof. Non-limiting examples of antibody variants include single-domain antibodies or nanobodies, monospecific Fab₂, bispecific Fab₂, trispecific Fab₃, monovalent IgGs, scFv, bispecific antibodies, bispecific diabodies, trispecific triabodies, scFv-Fc, minibodies, IgNAR, V-NAR, hcIgG, VhH, or peptibodies. A “peptibody” as provided herein refers to a peptide moiety attached (through a covalent or non-covalent linker) to the Fc domain of an antibody. Further non-limiting examples of antibody variants known in the art include antibodies produced by cartilaginous fish or camelids. A general description of antibodies from camelids and the variable regions thereof and methods for their production, isolation, and use may be found in references WO97/49805 and WO 97/49805 which are incorporated by reference herein in their entirety and for all purposes. Likewise, antibodies from cartilaginous fish and the variable regions thereof and methods for their production, isolation, and use may be found in WO2005/118629, which is incorporated by reference herein in its entirety and for all purposes.

The terms “CDR L1”, “CDR L2” and “CDR L3” as provided herein refer to the complementarity determining regions (CDR) 1, 2, and 3 of the variable light (L) chain of an antibody. In embodiments, the variable light chain provided herein includes in N-terminal to C-terminal direction a CDR L1, a CDR L2 and a CDR L3. Likewise, the terms “CDR H1”, “CDR H2” and “CDR H3” as provided herein refer to the complementarity determining regions (CDR) 1, 2, and 3 of the variable heavy (H) chain of an antibody. In embodiments, the variable light chain provided herein includes in N-terminal to C-terminal direction a CDR L1, a CDR L2 and a CDR L3.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e. fragment crystallizable region) is the “base” or “tail” of an immunoglobulin and is typically composed of two heavy chains that contribute two or three constant domains depending on the class of the antibody. By binding to specific proteins the Fc region ensures that each antibody generates an appropriate immune response for a given antigen. The Fc region also binds to various cell receptors, such as Fc receptors, and other immune molecules, such as complement proteins.

The term “antigen” as provided herein refers to molecules capable of binding to the antibody binding domain provided herein. An “antigen binding domain” as provided herein is a region of an antibody that binds to an antigen (epitope). As described above, the antigen binding domain is generally composed of one constant and one variable domain of each of the heavy and the light chain (VL, VH, CL and CH1, respectively). The paratope or antigen-binding site is formed on the N-terminus of the antigen binding domain. The two variable domains of an antigen binding domain typically bind the epitope on an antigen.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of an antibody is the region of its antigen to which the antibody binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

A “therapeutic agent” as referred to herein, is a composition useful in treating or preventing a disease such as cancer.

The phrase “specifically (or selectively) binds to an antibody” or “specifically (or selectively) immunoreactive with,” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions typically requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “ligand” refers to an agent, e.g., a polypeptide or other molecule, capable of binding to a receptor or antibody, antibody variant, antibody region or fragment thereof.

A “CD3 protein” as referred to herein includes any of the recombinant or naturally-occurring forms of the Cluster of Differentiation 3 (CD3) proteins or variants or homologs thereof that comprise the CD3 complex that mediates signal transduction and maintains CD3 complex activity (e.g. within at least 50%, 80%, 90%, 95%, 96%, 97%, 98%, 99% or 100% activity compared to the CD3 complex). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring CD3 proteins in the CD3 complex.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, that the resulting reaction product can be produced directly from a reaction between the added reagents or from an intermediate from one or more of the added reagents which can be produced in the reaction mixture.

The term “contacting” may include allowing two species to react, interact, or physically touch (e.g., bind), wherein the two species may be, for example, an antibody construct as described herein and a cancer protein. In embodiments, contacting includes, for example, allowing an antibody construct to bind to a cancer protein expressed on a cancer cell.

A “cell” as used herein, refers to a cell carrying out metabolic or other functions sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish. In some embodiments, the sample is obtained from a human.

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Patient” or “subject in need thereof” refers to a living organism suffering from or prone to a disease or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a patient is human.

The terms “disease” or “condition” refer to a state of being or health status of a patient or subject capable of being treated with a compound, pharmaceutical composition, or method provided herein. In embodiments, the disease is cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma (Mantel cell lymphoma), head and neck cancer, colorectal cancer, prostate cancer, pancreatic cancer, melanoma, breast cancer, neuroblastoma).

As used herein, the term “cancer” refers to all types of cancer, neoplasm or malignant tumors found in mammals, including leukemias, lymphomas, melanomas, neuroendocrine tumors, carcinomas and sarcomas. Exemplary cancers that may be treated with a compound, pharmaceutical composition, or method provided herein include lymphoma (e.g., Mantel cell lymphoma, follicular lymphoma, diffuse large B-cell lymphoma, marginal zona lymphoma, Burkitt's lymphoma), sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia (e.g., lymphoblastic leukemia, chronic lymphocytic leukemia, hairy cell leukemia), acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P388 leukemia model is widely accepted as being predictive of in vivo anti-leukemic activity. It is believed that a compound that tests positive in the P388 assay will generally exhibit some level of anti-leukemic activity in vivo regardless of the type of leukemia being treated. Accordingly, the present application includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

As used herein, the terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., breast, which site is referred to as a primary tumor, e.g., primary breast cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if lung cancer metastasizes to the breast, the secondary tumor at the site of the breast consists of abnormal lung cells and not abnormal breast cells. The secondary tumor in the breast is referred to a metastatic lung cancer. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors. For example, metastatic lung cancer refers to a disease in a subject with or with a history of a primary lung tumor and with one or more secondary tumors at a second location or multiple locations, e.g., in the breast.

The term “associated” or “associated with” in the context of a substance or substance activity or function associated with a disease (e.g., cancer (e.g. leukemia, lymphoma, B cell lymphoma, or multiple myeloma)) means that the disease (e.g. cancer, (e.g. leukemia, lymphoma, B cell lymphoma, or multiple myeloma)) is caused by (in whole or in part), or a symptom of the disease is caused by (in whole or in part) the substance or substance activity or function.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. For example, certain methods herein treat cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia lymphoblastic leukemia, chronic lymphocytic leukemia, hairy cell leukemia cancer cell), lymphoma (e.g., mantle cell lymphoma (MCL), follicular lymphoma, diffuse large B-cell lymphoma, marginal zone lymphoma, Burkitt's lymphoma), head and neck cancer, colorectal cancer, prostate cancer, pancreatic cancer, melanoma, breast cancer, neuroblastoma). For example certain methods herein treat cancer by decreasing or reducing or preventing the occurrence, growth, metastasis, or progression of cancer; or treat cancer by decreasing a symptom of cancer. Symptoms of cancer (e.g. lung cancer, ovarian cancer, osteosarcoma, bladder cancer, cervical cancer, liver cancer, kidney cancer, skin cancer (e.g., Merkel cell carcinoma), testicular cancer, leukemia, lymphoma, head and neck cancer, colorectal cancer, prostate cancer, pancreatic cancer, melanoma, breast cancer, neuroblastoma) would be known or may be determined by a person of ordinary skill in the art.

As used herein the terms “treatment,” “treat,” or “treating” refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

An “effective amount” is an amount sufficient to accomplish a stated purpose (e.g. achieve the effect for which it is administered, treat a disease, reduce enzyme activity, reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein relative to the absence of the antagonist. A “function disrupting amount,” as used herein, refers to the amount of antagonist required to disrupt the function of an enzyme or protein relative to the absence of the antagonist. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be coadministered to the patient. Coadministration is meant to include simultaneous or sequential administration of the compounds individually or in combination (more than one compound). Thus, the preparations can also be combined, when desired, with other active substances (e.g. to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

### Recombinant Protein Compositions

Provided herein are, inter alia, recombinant proteins (e.g., a chimeric antigen receptor, a bispecific antibody) for the treatment of cancer. The chimeric antigen receptor and bispecific antibody provided herein include a light chain variable domain capable of binding to human mantel cell lymphoma (MCL) cells. The recombinant proteins provided herein are, inter alia, useful for targeting and killing cancer cells while leaving healthy cells unharmed. The recombinant proteins provided herein, including embodiments thereof, are capable of specifically binding to human mantel cell lymphomas (MCLs) and causing targeted lysis of MCL cells in the presence of effector cells (e.g., NK cells). Surprisingly, the recombinant proteins provided herein do not bind to non-cancer (healthy) cells, thereby preventing adverse effects otherwise caused by unspecific killing of healthy cells. Due to their ability to differentially bind cancer cells versus non-cancer cells, the recombinant proteins provided herein are highly efficient and efficacious agents which may be used for therapeutic and diagnostic purposes. In embodiments, the recombinant protein is a chimeric antigen receptor (CAR). In embodiments, the recombinant protein is a bispecific antibody.

In an aspect is provided a recombinant protein including: (i) an antibody region including a light chain variable domain including a CDR L1 as set forth in SEQ ID NO:1, a CDR L2 as set forth in SEQ ID NO:2, and a CDR L3 as set forth in SEQ ID NO:3; and (ii) a transmembrane domain.

An “antibody region” as provided herein refers to a monovalent or multivalent protein moiety that forms part of the protein provided herein including embodiments thereof and that is capable of binding an antigen (epitope). The antibody region provided herein may include a domain of an antibody or fragment (e.g., Fab) thereof. Thus, the antibody region may include a light chain variable domain (VL) and/or a heavy chain variable domain (VH). In embodiments, the antibody region provided herein includes a light chain variable (VL) domain. In embodiments, the antibody region is a light chain variable (VL) domain.

As described above, a “light chain variable (VL) domain” as provided herein refers to a peptide (e.g., peptide domain) or peptidyl moiety capable of binding an antigen. A light chain variable (VL) domain as provided includes CDR sequences and framework region (FR) sequences of the light chain of an antibody, an antibody variant or fragment thereof. In embodiments, the light chain variable (VL) domain includes CDR L1 (SEQ ID NO:1), CDR L2 (SEQ ID NO:2), and CDR L3 (SEQ ID NO:3) of an antibody light chain. In embodiments, the light chain variable domain includes the sequence of SEQ ID NO:4. In embodiments, the light chain variable domain is the sequence of SEQ ID NO:4.

A “transmembrane domain” as provided herein refers to a polypeptide forming part of a biological membrane. The transmembrane domain provided herein is capable of spanning a biological membrane (e.g., a cellular membrane) from one side of the membrane through to the other side of the membrane. In embodiments, the transmembrane domain spans from the intracellular side to the extracellular side of a cellular membrane. Transmembrane domains may include non-polar, hydrophobic residues, which anchor the proteins provided herein including embodiments thereof in a biological membrane (e.g., cellular membrane of a T cell). Any transmembrane domain capable of anchoring the proteins provided herein including embodiments thereof are contemplated. Non-limiting examples of transmembrane domains include the transmembrane domains of CD28, CD8, CD4, CD3ζ, or CD8α. In embodiments, the transmembrane domain is a CD8α transmembrane domain, a CD28 transmembrane domain, a CD4 transmembrane domain, or a CD3 transmembrane domain. In embodiments, the transmembrane domain is a CD28 transmembrane domain. In embodiments, the transmembrane domain is a CD8 transmembrane domain. In embodiments, the transmembrane domain is a CD4 transmembrane domain. In embodiments, the transmembrane domain is a CD3 transmembrane domain. In embodiments, the transmembrane domain is a CD8α transmembrane domain. In embodiments, the CD8α transmembrane domain includes an amino acid sequence encoded by the sequence of SEQ ID NO:6. In embodiments, the CD8α transmembrane domain is an amino acid sequence encoded by the sequence of SEQ ID NO:6.

In embodiments, the recombinant protein as provided herein, including embodiments thereof, further includes an intracellular co-stimulatory signaling domain. An “intracellular co-stimulatory signaling domain” as provided herein includes amino acid sequences capable of providing co-stimulatory signaling in response to binding of an antigen to the antibody region provided herein including embodiments thereof. In embodiments, the signaling of the co-stimulatory signaling domain results in production of cytokines and proliferation of the T cell expressing the same. In embodiments, the intracellular co-stimulatory signaling domain is a CD28 intracellular co-stimulatory signaling domain, a 4-1BB intracellular co-stimulatory signaling domain, a ICOS intracellular co-stimulatory signaling domain, or an OX-40 intracellular co-stimulatory signaling domain. In embodiments, the intracellular co-stimulatory signaling domain is a CD28 intracellular co-stimulatory signaling domain. In embodiments, the intracellular co-stimulatory signaling domain is a ICOS intracellular co-stimulatory signaling domain. In embodiments, the intracellular co-stimulatory signaling domain is an OX-40 intracellular co-stimulatory signaling domain. In embodiments, the intracellular co-stimulatory signaling domain is a 4-1BB intracellular co-stimulatory signaling domain. In embodiments, the 4-1BB intracellular co-stimulatory signaling domain includes an amino acid sequence encoded by the sequence of SEQ ID NO: ______. In embodiments, the 4-1BB intracellular co-stimulatory signaling domain is an amino acid sequence encoded by the sequence of SEQ ID NO: ______.

In embodiments, the recombinant protein as provided herein including embodiments thereof, further includes an intracellular T-cell signaling domain. An “intracellular T-cell signaling domain” as provided herein includes amino acid sequences capable of providing primary signaling in response to binding of an antigen to the antibody region provided herein including embodiments thereof. In embodiments, the signaling of the intracellular T-cell signaling domain results in activation of the T cell expressing the same. In embodiments, the signaling of the intracellular T-cell signaling domain results in proliferation (cell division) of the T cell expressing the same. In embodiments, the signaling of the intracellular T-cell signaling domain results in expression by said T cell of proteins known in the art to be characteristic of activated T cells (e.g., CTLA-4, PD-1, CD28, CD69). In embodiments, the intracellular T-cell signaling domain includes the signaling domain of the zeta chain of the human CD3 complex. In embodiments, the intracellular T-cell signaling domain is a CD3ζ intracellular T-cell signaling domain. In embodiments, the CD3ζ intracellular T-cell signaling domain includes an amino acid sequence encoded by the sequence of SEQ ID NO ______. In embodiments, the CD3ζ intracellular T-cell signaling domain is an amino acid sequence encoded by the sequence of SEQ ID NO: ______.

The domains described above (e.g., light chain variable domain, transmembrane domain, intracellular co-stimulatory domain, intracellular T-cell signaling domain) can have a specific order in the recombinant protein from the N-terminus to the C-terminus. Thus, in embodiments, from the N-terminus to the C-terminus, the recombinant protein includes a light chain variable domain as provided herein including embodiments thereof, a transmembrane domain as provided herein including embodiments thereof, an intracellular co-stimulatory domain as provided herein including embodiments thereof, and a intracellular T-cell signaling domain as provided herein including embodiments thereof. In one embodiment, from the N-terminus to the C-terminus, the recombinant protein includes a light chain variable domain of SEQ ID NO:4, a CD8α transmembrane domain encoded by SEQ ID NO:6, a 4-1BB intracellular co-stimulatory domain encoded by SEQ ID NO:7, and a CD3ζ intracellular T-cell signaling domain encoded by SEQ ID NO:8.

In embodiments, the recombinant protein provided herein, including embodiments thereof, further includes a detectable domain. A “detectable domain” as provided herein is a peptide moiety detectable by spectroscopic, photochemical, biochemical, immunochemical, chemical, or other physical means. For example, a detectable domain as provided herein may be a protein or other entity which can be made detectable, e.g., by incorporating a radiolabel or being reactive to an antibody specifically. Any appropriate method known in the art for conjugating an antibody to the label may be employed, e.g., using methods described in Hermanson, Bioconjugate Techniques 1996, Academic Press, Inc., San Diego. In the present invention, a detectable domain is used to confirm transfection of T cells.

In embodiments, detectable domain is a fluorescent protein. In embodiments, the detectable domain is EGFP. In embodiments, detectable domain is a truncated EGFR (EGFRt) domain. The term “EGFRt” refers to a truncated epidermal growth factor receptor protein lacking intracellular signaling capabilities. The EGFRt contains only the transmembrane domain, Domain III, and Domain IV of wild-type human EGFR.

As used herein, EGFRt is an inert cell surface molecule which functions as a detectable domain to identify T cells transduced with a nucleic acid encoding the CAR polypeptide provided herein or individual domains thereof (e.g., light chain variable domain, transmembrane domain, intracellular co-stimulatory domain, intracellular T-cell signaling domain). The detectable domain (e.g., EGFRt) may form part of the recombinant protein provided herein (CAR polypeptide) or it may be present as a separate protein, not forming part of the CAR polypeptide. Thus, the detectable domain (e.g., EGFRt) may be encoded by a nucleic acid sequence that forms part of the same nucleic acid encoding the CAR polypeptide provided herein or individual domains thereof (e.g., light chain variable domain, transmembrane domain, intracellular co-stimulatory domain, intracellular T-cell signaling domain) or the detectable domain (e.g., EGFRt) may be encoded by a separate nucleic acid sequence. Where the detectable domain (e.g., EGFRt) is encoded by a nucleic acid sequence forming part or the same nucleic acid encoding the CAR polypeptide domains, it may be translated from an open reading frame distinct from the open reading frame encoding the CAR domains.

In embodiments, the recombinant protein binds to a cell. In embodiments, the cell is a cancer cell. In embodiments, the cancer cell is a lymphoma, leukemia, or myeloma cancer cell. In embodiments, the cancer cell is a lymphoma cancer cell. In embodiments, the cancer cell is a leukemia cancer cell. In embodiments, the cancer cell is a myeloma cancer cell.

In embodiments, the lymphoma cancer cell is a non-Hodgkin's lymphoma cancer cell. Non-Hodgkin's lymphoma is a type of cancer that originates in the lymphatic system. Non-Hodgkin's lymphoma tumors develop from a type of small leukocyte (i.e., white blood cell) known as a lymphocyte. In embodiments, the non-Hodgkin's lymphoma cancer cell is a mantle cell lymphoma (MCL), follicular lymphoma, diffuse large B-cell lymphoma, marginal zone lymphoma, or Burkitt's lymphoma cancer cell. In embodiments, the non-Hodgkin's lymphoma cancer cell is a mantle cell lymphoma (MCL) cancer cell. In embodiments, the non-Hodgkin's lymphoma cancer cell is a follicular lymphoma cancer cell. In embodiments, the non-Hodgkin's lymphoma cancer cell is a diffuse large B-cell lymphoma cancer cell. In embodiments, the non-Hodgkin's lymphoma cancer cell is a marginal zone lymphoma cancer cell. In embodiments, the non-Hodgkin's lymphoma cancer cell is a Burkitt's lymphoma cancer cell.

In embodiments, the leukemia cancer cell is a lymphoblastic leukemia, chronic lymphocytic leukemia, or hairy cell leukemia cancer cell. In embodiments, the leukemia cancer cell is a lymphoblastic leukemia cancer cell. In embodiments, the leukemia cancer cell is a chronic lymphocytic leukemia cancer cell. In embodiments, the leukemia cancer cell is a hairy cell leukemia cancer cell.

In embodiments, the recombinant protein forms part of a cell. In embodiments, the recombinant protein forms part of a T cell. In embodiments, the recombinant protein forms part of a CD4+ T cell. In embodiments, the recombinant protein forms part of a CD8+ T cell.

The light chain variable (VL) domain provided herein may form part of a bispecific antibody. Thus, in an aspect is provided a recombinant protein including: (i) a first antibody region capable of binding an effector cell ligand; and (ii) a second antibody region, including a light chain variable domain including a CDR L1 as set forth in SEQ ID NO:1, a CDR L2 as set forth in SEQ ID NO:2 and a CDR L3 as set forth in SEQ ID NO:3. In embodiments, the light chain variable domain includes the sequence of SEQ ID NO:4.

The term “effector cell ligand” as provided herein refers to a cell surface molecule expressed on an effector cell of the immune system (e.g., a cytotoxic T cell, a helper T cell, a B cell, a natural killer cell). Upon binding of the first antibody region to the effector cell ligand expressed on the effector cell, the effector cell is activated and able to exert its function (e.g., selective killing or eradication of malignant, infected or otherwise unhealthy cells). In embodiments, the effector cell ligand is a CD3 protein.

The first antibody region as provided herein may be an antibody, an antibody variant, a fragment of an antibody, or a fragment of an antibody variant. In embodiments, the first antibody region is a Fab′ fragment. In embodiments, the first antibody region is a single chain variable fragment (scFv). In embodiments, the scFv includes an amino acid sequence encoded by the sequence of SEQ ID NO:10. In embodiments, the scFv is an amino acid sequence encoded by the sequence of SEQ ID NO:10.

In embodiments, the recombinant protein provided herein, including embodiments thereof, further includes a first constant heavy chain 3 (CH3) domain bound to the first antibody region through a first constant heavy chain 2 (CH2) domain, and a second constant heavy chain 3 (CH3) domain bound to the second antibody region through a second constant heavy chain 2 (CH2) domain. In embodiments, the first constant heavy chain 3 (CH3) domain and the first constant heavy chain 2 (CH2) form part of the sequence of SEQ ID NO:12. In embodiments, the second constant heavy chain 3 (CH3) domain and the second constant heavy chain 2 (CH2) form part of the sequence of SEQ ID NO:11. In embodiments, the first constant heavy chain 3 (CH3) domain and the first constant heavy chain 2 (CH2) are the polypeptide of the sequence of SEQ ID NO:12. In embodiments, the second constant heavy chain 3 (CH3) domain and the second constant heavy chain 2 (CH2) are the polypeptide of the sequence of SEQ ID NO:11.

In embodiments, the first CH3 domain is bound to the second CH3 domain.

In embodiments, the recombinant protein binds to a cell. In embodiments, the cell is a cancer cell. In embodiments, the cancer cell is a lymphoma, leukemia, or myeloma cancer cell. In embodiments, the cancer cell is a lymphoma. In embodiments, the cancer cell is a leukemia cancer cell. In embodiments, the cancer cell is a myeloma cancer cell.

In embodiments, the lymphoma cancer cell is a non-Hodgkin's lymphoma cancer cell. In embodiments, the non-Hodgkin's lymphoma cancer cell is a mantle cell lymphoma (MCL), follicular lymphoma, diffuse large B-cell lymphoma, marginal zone lymphoma, or Burkitt's lymphoma cancer cell. In embodiments, the non-Hodgkin's lymphoma cancer cell is a mantle cell lymphoma (MCL) cancer cell. In embodiments, the non-Hodgkin's lymphoma cancer cell is a follicular lymphoma cancer cell. In embodiments, the non-Hodgkin's lymphoma cancer cell is a diffuse large B-cell lymphoma cancer cell. In embodiments, the non-Hodgkin's lymphoma cancer cell is a marginal zone lymphoma cancer cell. In embodiments, the non-Hodgkin's lymphoma cancer cell is a Burkitt's lymphoma cancer cell.

In embodiments, the leukemia cancer cell is a lymphoblastic leukemia, chronic lymphocytic leukemia, or hairy cell leukemia cancer cell. In embodiments, the leukemia cancer cell is a lymphoblastic leukemia cancer cell. In embodiments, the leukemia cancer cell is a chronic lymphocytic leukemia cancer cell. In embodiments, the leukemia cancer cell is a hairy cell leukemia cancer cell.

translated from an open reading frame distinct from the open reading frame encoding the CAR domains.

In embodiments, the recombinant protein binds to a cell. In embodiments, the cell is a cancer cell. In embodiments, the cancer cell is a lymphoma, leukemia, or myeloma cancer cell. In embodiments, the cancer cell is a lymphoma cancer cell. In embodiments, the cancer cell is a leukemia cancer cell. In embodiments, the cancer cell is a myeloma cancer cell.

In embodiments, the lymphoma cancer cell is a non-Hodgkin's lymphoma cancer cell. Non-Hodgkin's lymphoma is a type of cancer that originates in the lymphatic system. Non-Hodgkin's lymphoma tumors develop from a type of small leukocyte (i.e., white blood cell) known as a lymphocyte. In embodiments, the non-Hodgkin's lymphoma cancer cell is a mantle cell lymphoma (MCL), follicular lymphoma, diffuse large B-cell lymphoma, marginal zone lymphoma, or Burkitt's lymphoma cancer cell. In embodiments, the non-Hodgkin's lymphoma cancer cell is a mantle cell lymphoma (MCL) cancer cell. In embodiments, the non-Hodgkin's lymphoma cancer cell is a follicular lymphoma cancer cell. In embodiments, the non-Hodgkin's lymphoma cancer cell is a diffuse large B-cell lymphoma cancer cell. In embodiments, the non-Hodgkin's lymphoma cancer cell is a marginal zone lymphoma cancer cell. In embodiments, the non-Hodgkin's lymphoma cancer cell is a Burkitt's lymphoma cancer cell.

In embodiments, the leukemia cancer cell is a lymphoblastic leukemia, chronic lymphocytic leukemia, or hairy cell leukemia cancer cell. In embodiments, the leukemia cancer cell is a lymphoblastic leukemia cancer cell. In embodiments, the leukemia cancer cell is a chronic lymphocytic leukemia cancer cell. In embodiments, the leukemia cancer cell is a hairy cell leukemia cancer cell.

In embodiments, the recombinant protein forms part of a cell. In embodiments, the recombinant protein forms part of a T cell. In embodiments, the recombinant protein forms part of a CD4+ T cell. In embodiments, the recombinant protein forms part of a CD8+ T cell.

The light chain variable (VL) domain provided herein may form part of a bispecific antibody. Thus, in an aspect is provided a recombinant protein including: (i) a first antibody region capable of binding an effector cell ligand; and (ii) a second antibody region, including a light chain variable domain including a CDR L1 as set forth in SEQ ID NO:1, a CDR L2 as set forth in SEQ ID NO:2 and a CDR L3 as set forth in SEQ ID NO:3. In embodiments, the light chain variable domain includes the sequence of SEQ ID NO:4.

The term “effector cell ligand” as provided herein refers to a cell surface molecule expressed on an effector cell of the immune system (e.g., a cytotoxic T cell, a helper T cell, a B cell, a natural killer cell). Upon binding of the first antibody region to the effector cell ligand expressed on the effector cell, the effector cell is activated and able to exert its function (e.g., selective killing or eradication of malignant, infected or otherwise unhealthy cells). In embodiments, the effector cell ligand is a CD3 protein.

The first antibody region as provided herein may be an antibody, an antibody variant, a fragment of an antibody, or a fragment of an antibody variant. In embodiments, the first antibody region is a Fab′ fragment. In embodiments, the first antibody region is a single chain variable fragment (scFv). In embodiments, the scFv includes an amino acid sequence encoded by the sequence of SEQ ID NO:10. In embodiments, the scFv is an amino acid sequence encoded by the sequence of SEQ ID NO:10.

In embodiments, the recombinant protein provided herein, including embodiments thereof, further includes a first constant heavy chain 3 (CH3) domain bound to the first antibody region through a first constant heavy chain 2 (CH2) domain, and a second constant heavy chain 3 (CH3) domain bound to the second antibody region through a second constant heavy chain 2 (CH2) domain. In embodiments, the first constant heavy chain 3 (CH3) domain and the first constant heavy chain 2 (CH2) form part of the sequence of SEQ ID NO:12. In embodiments, the second constant heavy chain 3 (CH3) domain and the second constant heavy chain 2 (CH2) form part of the sequence of SEQ ID NO:11. In embodiments, the first constant heavy chain 3 (CH3) domain and the first constant heavy chain 2 (CH2) are the polypeptide of the sequence of SEQ ID NO:12. In embodiments, the second constant heavy chain 3 (CH3) domain and the second constant heavy chain 2 (CH2) are the polypeptide of the sequence of SEQ ID NO:11.

In embodiments, the first CH3 domain is bound to the second CH3 domain.

In embodiments, the recombinant protein binds to a cell. In embodiments, the cell is a cancer cell. In embodiments, the cancer cell is a lymphoma, leukemia, or myeloma cancer cell. In embodiments, the cancer cell is a lymphoma. In embodiments, the cancer cell is a leukemia cancer cell. In embodiments, the cancer cell is a myeloma cancer cell.

In embodiments, the lymphoma cancer cell is a non-Hodgkin's lymphoma cancer cell. In embodiments, the non-Hodgkin's lymphoma cancer cell is a mantle cell lymphoma (MCL), follicular lymphoma, diffuse large B-cell lymphoma, marginal zone lymphoma, or Burkitt's lymphoma cancer cell. In embodiments, the non-Hodgkin's lymphoma cancer cell is a mantle cell lymphoma (MCL) cancer cell. In embodiments, the non-Hodgkin's lymphoma cancer cell is a follicular lymphoma cancer cell. In embodiments, the non-Hodgkin's lymphoma cancer cell is a diffuse large B-cell lymphoma cancer cell. In embodiments, the non-Hodgkin's lymphoma cancer cell is a marginal zone lymphoma cancer cell. In embodiments, the non-Hodgkin's lymphoma cancer cell is a Burkitt's lymphoma cancer cell.

In embodiments, the leukemia cancer cell is a lymphoblastic leukemia, chronic lymphocytic leukemia, or hairy cell leukemia cancer cell. In embodiments, the leukemia cancer cell is a lymphoblastic leukemia cancer cell. In embodiments, the leukemia cancer cell is a chronic lymphocytic leukemia cancer cell. In embodiments, the leukemia cancer cell is a hairy cell leukemia cancer cell.

### BRIEF DESCRIPTION OF THE DRAWINGS

FIGS. 1A-C. Identification and characterization of mantle cell lymphoma-specific antibody phage clones. FIG. 1A: Phages bound to JeKo cells were eluted, titered and amplified for the next round of biopanning. Biopanning enrichment was expressed in phage “Output/input ratio” (×10⁻⁸) or “Number of phages/10⁶ cells”. FIG. 1B: Predominant JeKo-binding phage clones were generated and analyzed for their binding specificity. Phage clones were titrated and staining on JeKo cells was compared to L cells (negative control). Unstained cells and secondary antibody alone were used as additional negative controls to exclude non-specific binding. The data are representative of three independent experiments. FIG. 1C: C4-4 binding specificity was examined based on the staining of different lymphoma cell lines, including mantle cell lymphoma cell lines—JeKo, Mino, Maver and NCEB over HBL1 diffused large b-cell lymphoma (DLBCL), RL follicular lymphoma (FL), Raji and BJAB burkitt's lymphoma cell lines (BL). C4-4 phage clone was titrated; unstained cells and secondary antibody alone were used as negative controls to exclude non-specific binding.

FIG. 2. Schematic representation of 4-4 chimeric antigen receptor (2^{nd} generation CAR).

FIGS. 3A-3C. Tumor-specific cytotoxicity of 4-4 CAR T cells in vitro. FIG. 3A: In vitro cytotoxicity assay of 4-4 pan T CARs or non-transduced control T cells cultured with JeKo-1 cell line or normal B cells at the indicated effector-to-target ratios. Chromium (50Cr) release was measured as a readout of cytotoxicity. FIG. 3B: In vitro cytotoxicity assay of 4-4 CD8 CARs or non-transduced control T cells (Non CAR T), cultured with JeKo-1 cell line or normal B cells at the indicated effector-to-target ratios. Chromium (50Cr) release was measured as a readout of cytotoxicity. FIG. 3C: The specificity of 4-4 CAR T cell cytotoxicity was tested on JeKo, Mino, Z138, REC-1, Maver and NCEB-1 mantle cell lymphoma cell lines, RL follicular lymphoma, RS4 acute lymphocytic leukemia and Raji burkitt's lymphoma cell line. Cytotoxicity was induced in the presence of target cells with pan T CARs or CD8 CARs for 4 hours. The effector to target cell ratio was 10:1.

FIGS. 4A-4B. Generation of 4-4 Bispecific Antibody by Knob-Hole Technology. FIG. 4A: Schematic presentation of bispecific design. ScFv targeting CD3 and light chain variable region targeting MCL were fused with human Ig1 Fc fragments. Linkers were constructed between the VH and VL domains of their respective scFvs and consisted of (Gly4Se4)₃. Knobs-into-holes construct was applied in order to facilitate the heterodimer formation. FIG. 4B: Expression of bispecific antibody.

FIGS. 5A-5C. Tumor-specific cytotoxicity of 4-4 BITE in vitro. FIG. 5A: Cytotoxicity of 4-4 BITE on Jeko cells. In vitro cytotoxicity assay of 4-4 bispecific T cell engager (BiTE) on Jeko cell line and normal B cells at the indicated concentrations. Cytotoxicity was induced in the presence of activated T cells with an effector to target cell ratio of 10:1 for 4 hours. Chromium (50Cr) release was measured as a readout of cytotoxicity. FIG. 5B: Cytotoxicity of 4-4 BITE on Jeko cells. In vitro cytotoxicity assay of 4-4 bispecific T cell engager (BiTE) on Jeko cell line at the indicated effector-to-target ratios. Chromium (50Cr) release was measured as a readout of cytotoxicity. FIG. 5C: Mantle cell-specific cytotoxicity of 4-4 Bispecific Ab. The specificity of 4-4 BITE was tested on JeKo, Mino, Z138, REC-1, Maver and NCEB-1 mantle cell lymphoma cell lines, RL follicular lymphoma, RS4 acute lymphocytic leukemia and Raji burkitt's lymphoma cell line. Cytotoxicity was induced in the presence of target cells with activated pan T cells or CD8N cells for 4 hours. The effector to target cell ratio was 10:1. Antibody concentration was 2 ug for 10⁶ target cells.

FIG. 6. Cytokine-release assays. Cytokine-release assays of mantle cell lymphoma-specific CAR and BITE. Analysis of interferon-gamma (IFN-gamma) and tumor necrosis factor-a (TNFa) release from CAR T cells, BITE+ T cells and non-transduced control T cells, cultured with JEKO, Mino, Z138, and normal B cells.

FIG. 7. Elements included in the CAR construct, order of elements, sequences of the individual elements, and schematic of vector.

FIGS. 8A-8B. Tumor-specific CAR T-cell cytokine release and degranulation. ELISA measurements of (A) cytokines: IL2, TNF-α, and INF-γ; or (B) granules: perforin and granzyme; from supernatant of MCL (4-4) CD4 or CD8 CAR T cells incubated with various tumor lines or normal B cells. Non-CAR transduced T cells from the same donor was used as controls for each assay.

FIGS. 9A-9B. In vivo MCL (4-4) CAR T-cell treatment. (A) Bioluminescence images of groups of NSG mice (n=5) following intravenous (IV) tumor challenge (5×104 cells/mouse) on day 0 with luciferase-expressing Z-138. Activated CAR T-cell treatments were infused by IV on day 11. Treatments consisted of 2.5×106 CD4+TN+1×106 CD8+TN CAR-T cells. Non-transduced (control) T cells (3.5×106 cells) from the same donor or PBS was used as controls. (B) Kaplan-Meier plot of overall survival over the course of 75

days. **P<0.001

FIGS. 10A-10B. In vivo MCL (4-4) BiTE antibody treatment. (A) Bioluminescence images of groups of NSG mice (n=5) following intravenous (IV) tumor challenge (5×104 cells/mouse) on day 0 with luciferase-expressing Z-138. Three treatments given on days 4, 8, and 12 consisted of 400 μg MCL (4-4) antibody+10×106 isolated PBMC. PBMC from the same donor alone or PBS were used as controls. (B) Kaplan-Meier plot of overall survival over the course of 70 days. **P<0.001

FIGS. 11A-11B. In vivo MCL (4-4) BiTE antibody treatment. (A) Bioluminescence images of groups of NSG mice (n=5) following intravenous (IV) tumor challenge (5×104 cells/mouse) on day 0 with luciferase-expressing Z-138. Four treatments given on days 11, 14, 17, and 20 consisted of 200 μg or 400 μg MCL (4-4) antibody+5×106 isolated pan T cells. T cells from the same donor alone or PBS were used as controls. (B) Kaplan-Meier plot of overall survival over the course of 90 days. **P<0.001

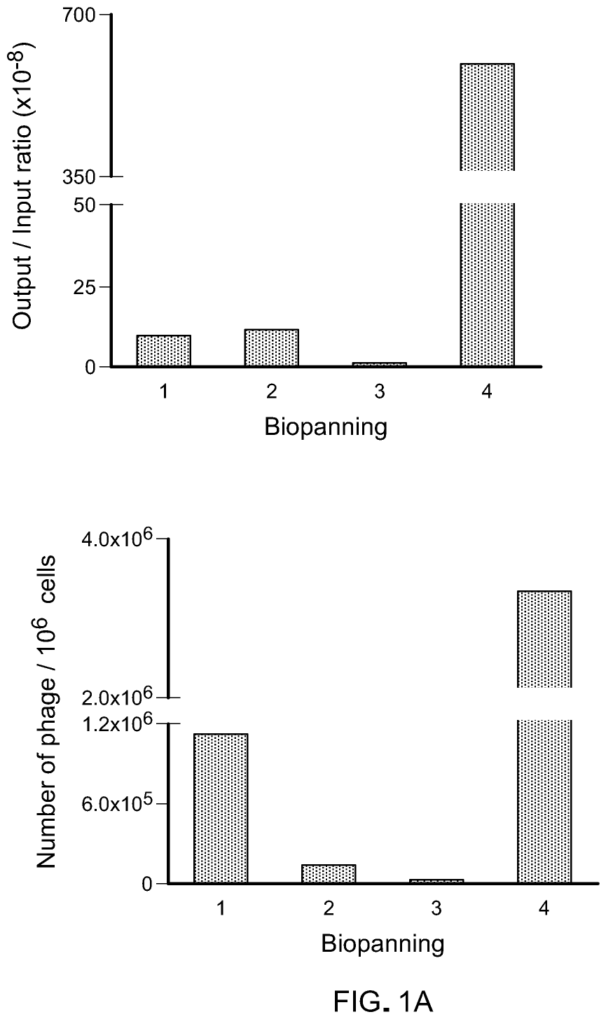

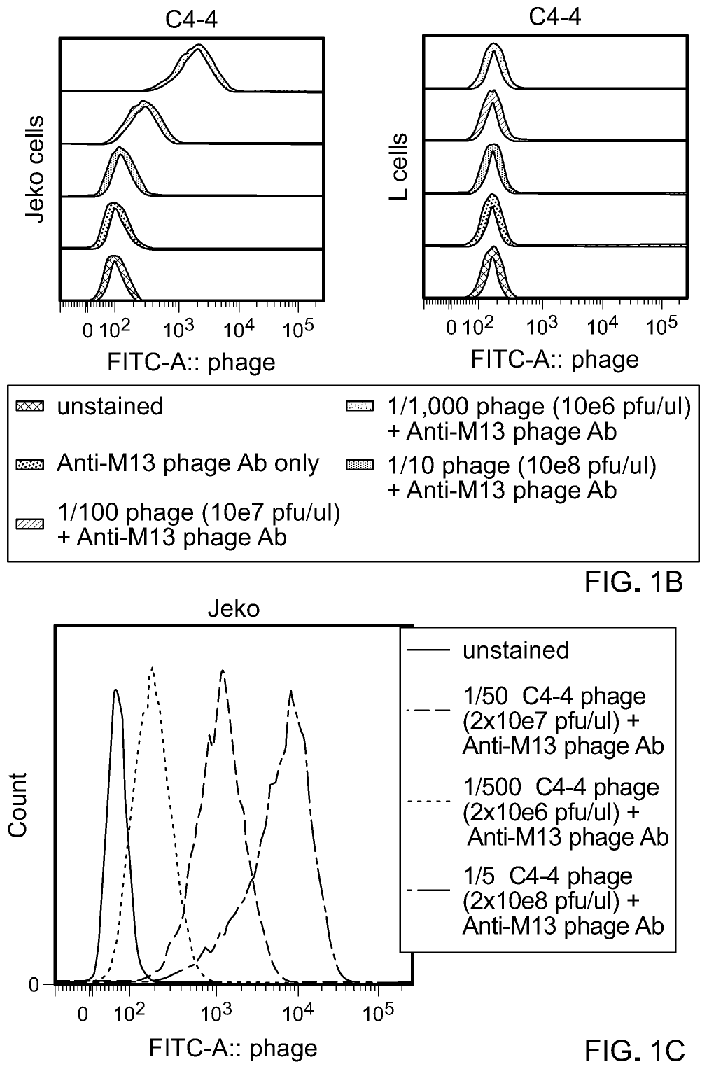

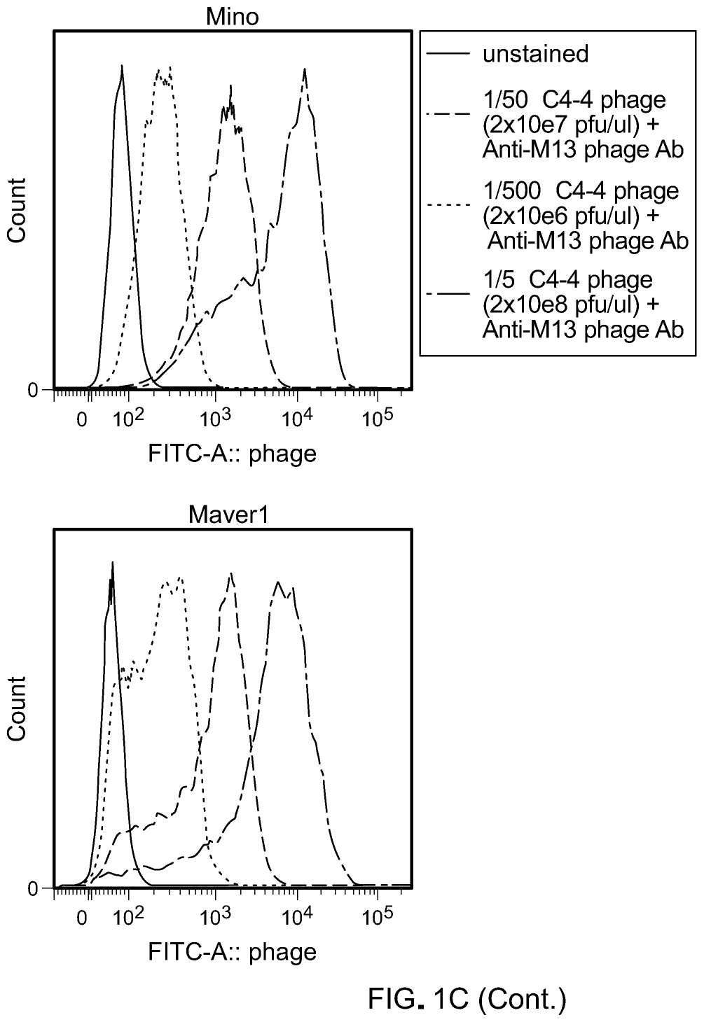

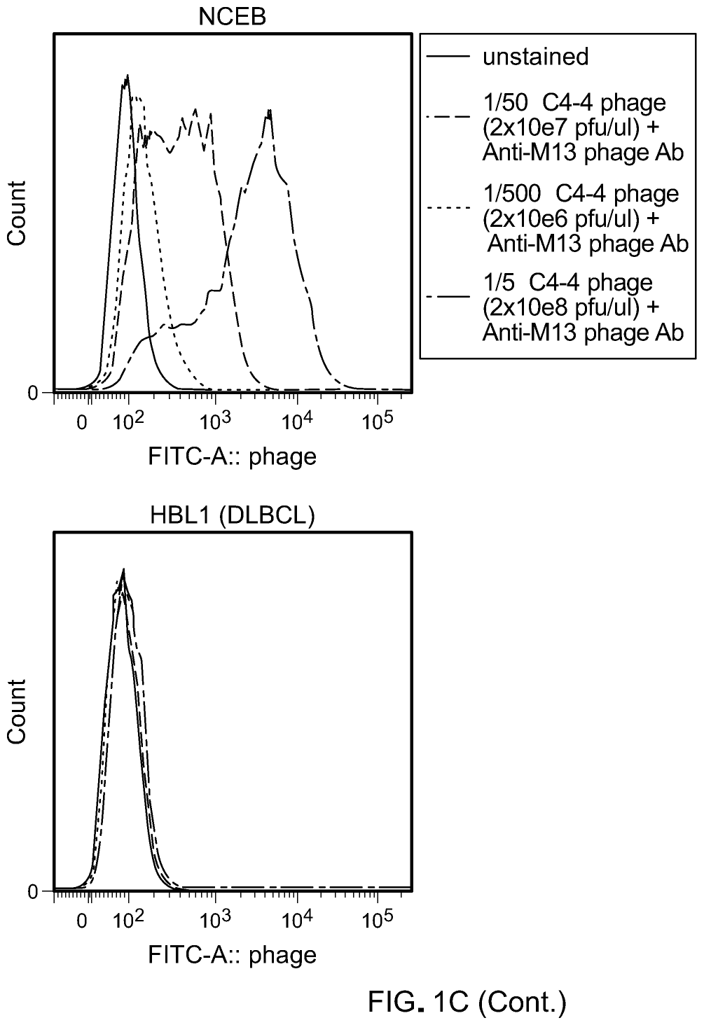

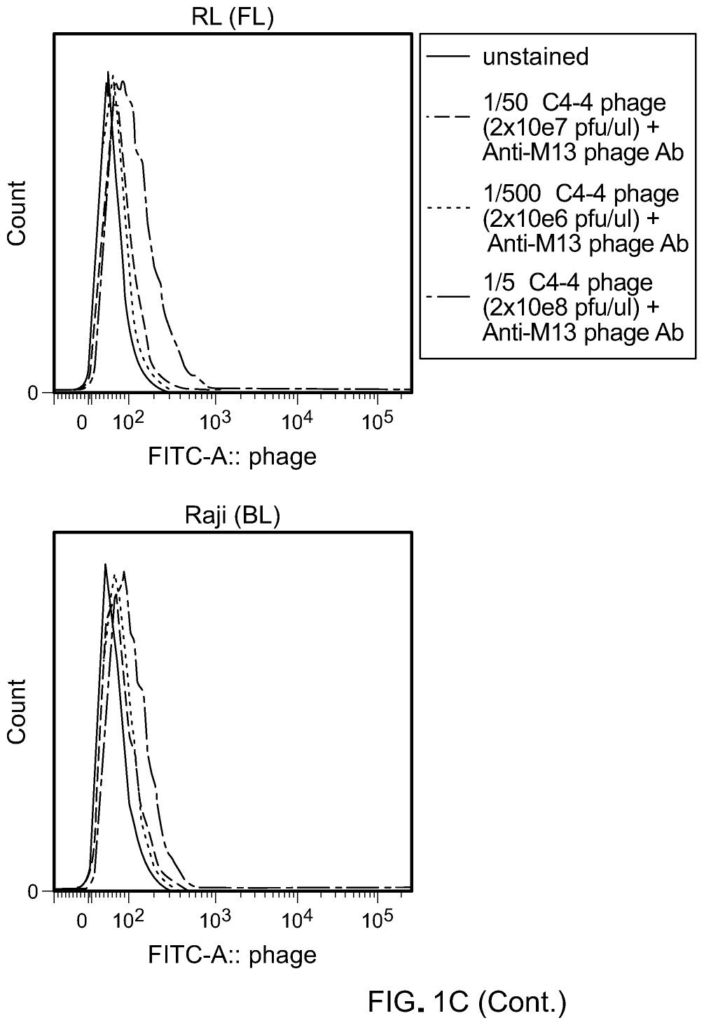

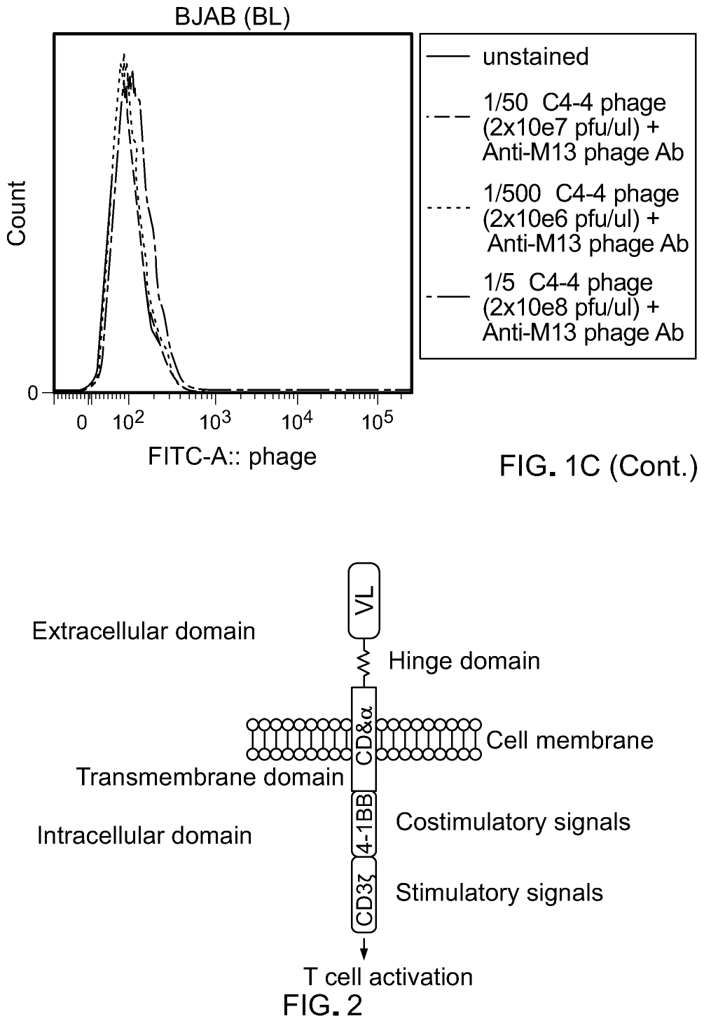

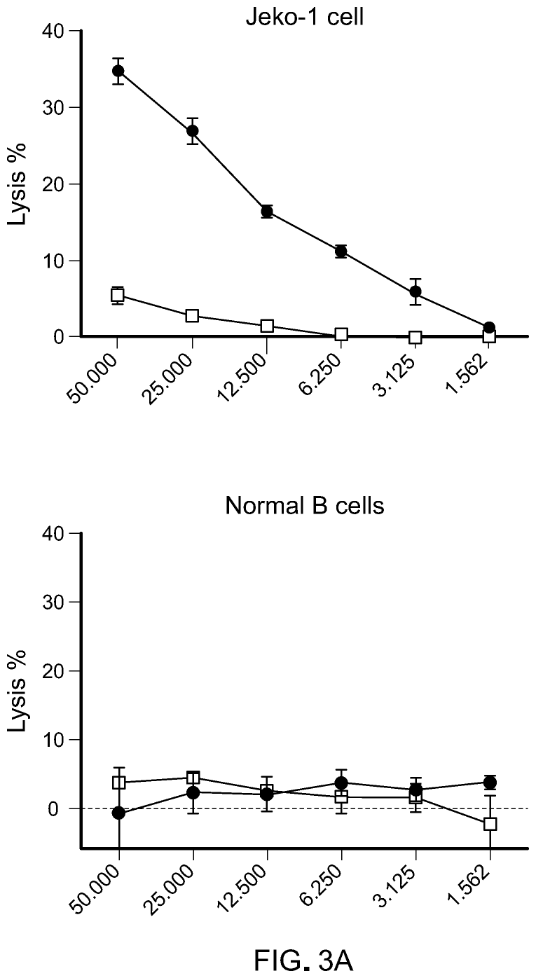

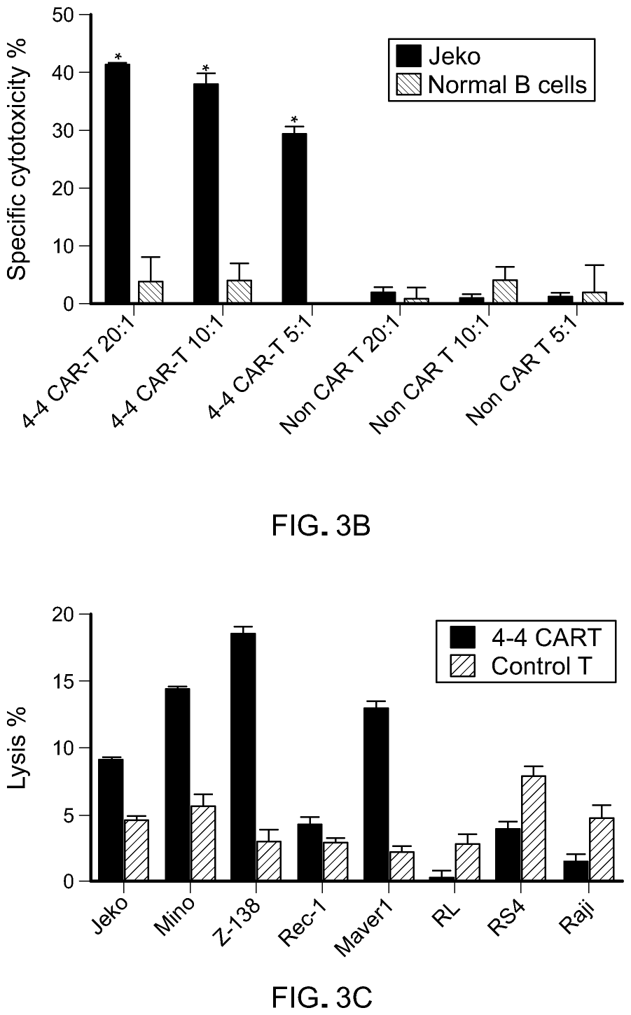

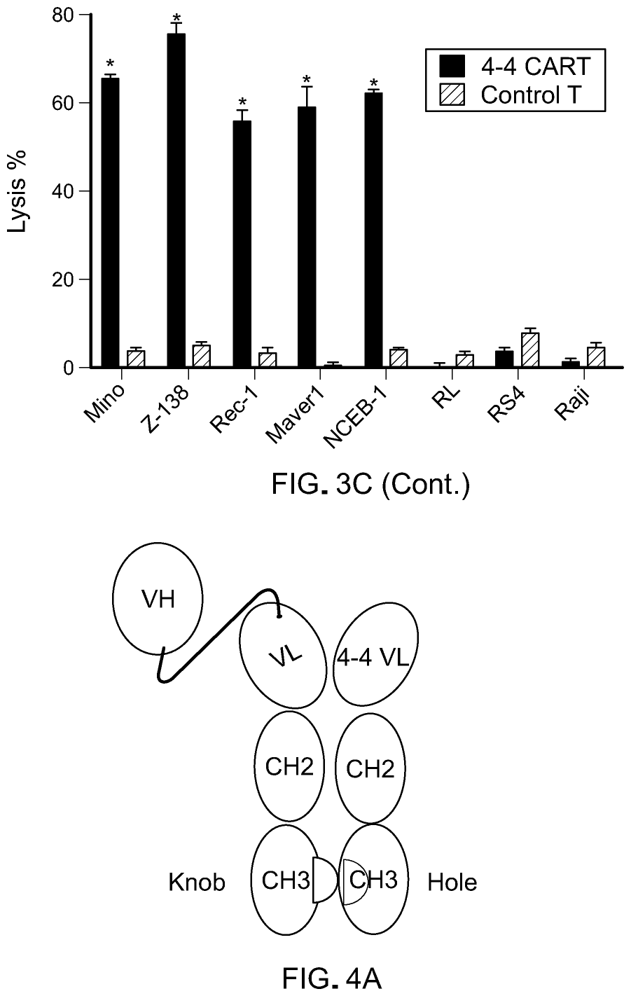

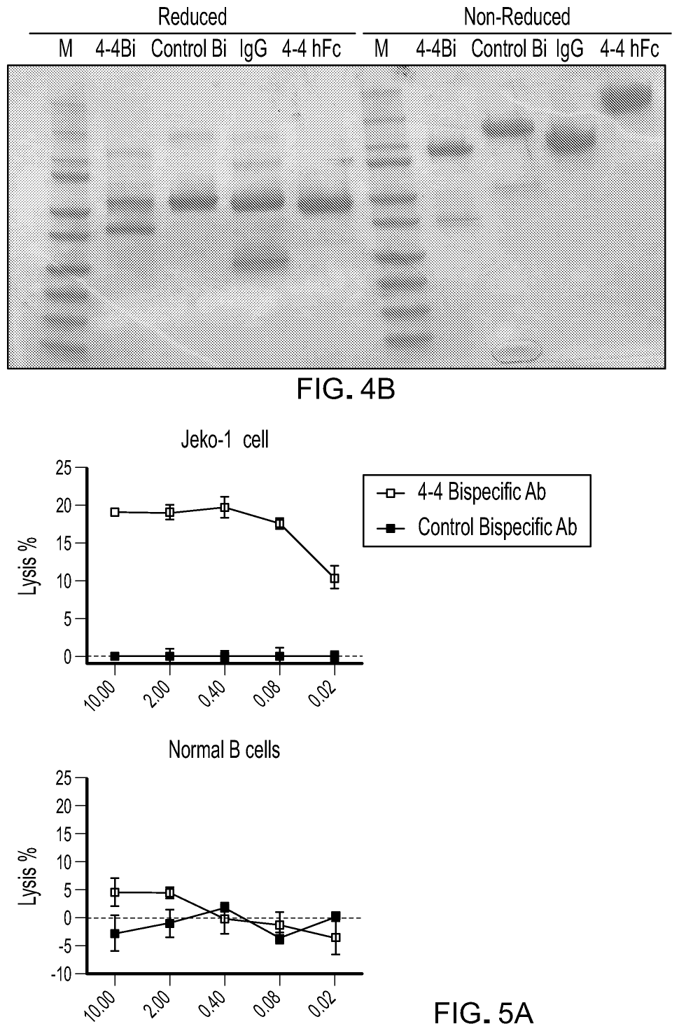

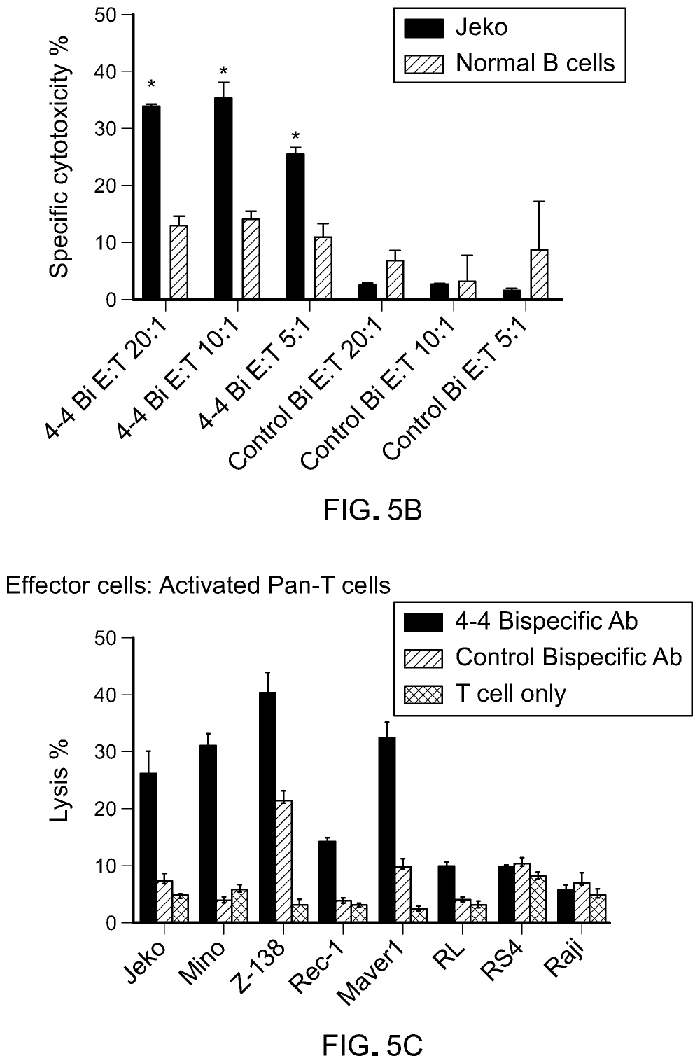

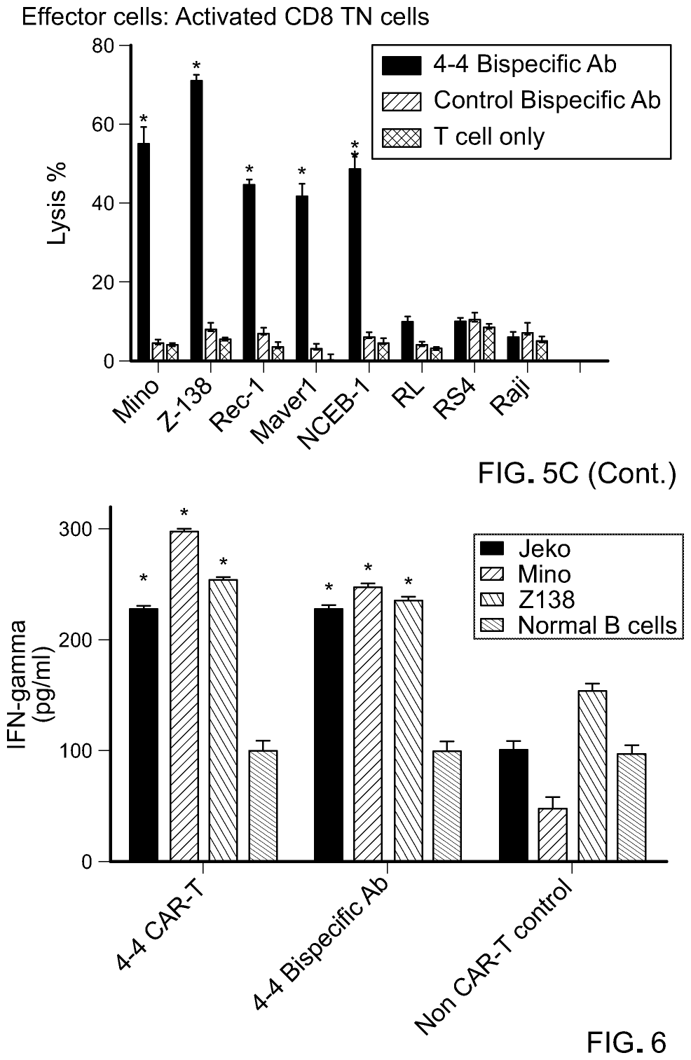

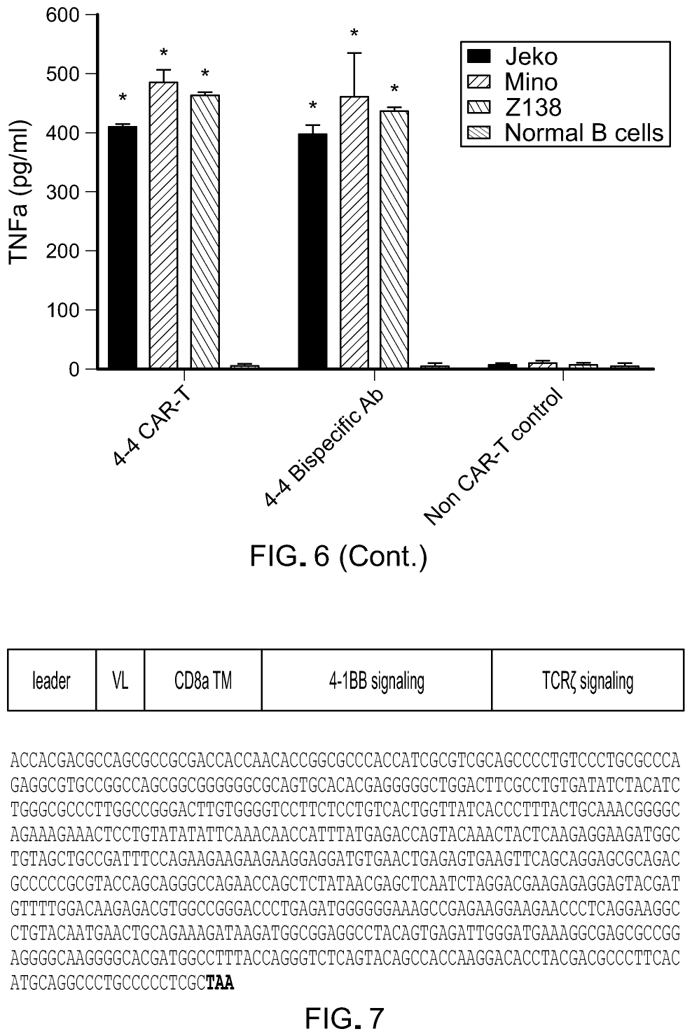

### DETAILED DESCRIPTION

### Chimeric Antigen Receptors

### Spacer Region

The CAR described herein can include a spacer located between the targeting domain and the transmembrane domain. A variety of different spacers can be used. Some of them include at least portion of a human Fc region, for example a hinge portion of a human Fc region or a CH3 domain or variants thereof. Table 1 below provides various spacers that can be used in the CARs described herein.

TABLE 1
Examples of Spacers

| Name | Length | Sequence |
|---|---|---|
| a3 | 3 aa | AAA |
| linker | 10 aa | GGGSSGGGSG (SEQ ID NO: _) |
| IgG4 hinge (S→P) | 12 aa | ESKYGPPCPPCP (SEQ ID NO: _) |
| (S228P) | | |
| IgG4 hinge | 12 aa | ESKYGPPCPSCP (SEQ ID NO: -) |
| IgG4 hinge (S228P) + linker | 22 aa | ESKYGPPCPPCPGGGSSGGGSG (SEQ ID NO: -) |
| CD28 hinge | 39 aa | IEVMYPPPYLDNEKSNGTIIHVKGKHLCPSPLFPGPSKP (SEQ ID NO: -) |
| CD8 hinge-48 aa | 48 aa | AKPTTTPAPRPPTPAPTIASQPLSLRPEACRPAAGGAVHTRGLDFACD |
| | | (SEQ ID NO: -) |
| CD8 hinge-45 aa | 45 aa | TTTPAPRPPTPAPTIASQPLSLRPEACRPAAGGAVHTRGLDFACD |
| | | (SEQ ID NO: -) |
| IgG4 (HL-CH3) | 129 aa | ESKYGPPCPPCPGGGSSGGGSGGQPREPQVYTLPPSQEEMTKNQVSLTCL |
| (includes S228P in hinge) | | VKGFYPSDIAVEWESNGQPENNYKTTPPVLDSDGSFFLYSRLTVDKSRWQE |
| | | GNVFSCSVMHEALHNHYTQKSLSLSLGK (SEQ ID NO: 9) |
| IgG4 (L235E, N297Q) | 229 aa | ESKYGPPCPSCPAPEFEGGPSVFLFPPKPKDTLMISRTPEVTCVVVDVSQED |
| | | PEVQFNWYVDGVEVHQAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: -) |
| IgG4 (S228P, L235E, N297Q) | 229 aa | ESKYGPPCPPCPAPEFEGGPSVFLFPPKPKDTLMISRTPEVTCVVVDVSQED |
| | | PEVQFNWYVDGVEVHQAKTKPREEQFQSTYRVVSVLTVLHQDWLNGKEY |
| | | KCKVSNKGLPSSIEKTISKAKGQPREPQVYTLPPSQEEMTKNQVSLTCLVKG |
| | | FYPSDIAVEWESNGQPENNYKTTPPVLDSDGSFFLYSRLTVDKSRWQEGN |
| | | VFSCSVMHEALHNHYTQKSLSLSLGK (SEQ ID NO: -) |
| IgG4 (CH3) | 107 aa | GQPREPQVYTLPPSQEEMTKNQVSLTCLVKGFYPSDIAVEWESNGQPENN |
| | | YKTTPPVLDSDGSFFLYSRLIVDKSRWQEGNVFSCSVMHEALHNHYTQKSL |
| | | SLSLGK (SEQ ID NO: -) |

Some spacer regions include all or part of animmunoglobulin (e.g., IgG1, IgG2, IgG3, IgG4) hinge region, i.e., the sequence that falls between the CH1 and CH2 domains of an immunoglobulin, e.g., an IgG4 Fc hinge or a CD8 hinge. Some spacer regions include an immunoglobulin CH3 domain or both a CH3 domain and a CH2 domain. The immunoglobulin derived sequences can include one or more amino acid modifications, for example, 1, 2, 3, 4 or 5 substitutions, e.g., substitutions that reduce off-target binding.

The hinge/linker region can also comprise a IgG4 hinge region having the sequence ESKYGPPCPSCP (SEQ ID NO: ______) or ESKYGPPCPPCP (SEQ ID NO: ______).

The hinge/linger region can also comprise the sequence ESKYGPPCPPCP (SEQ ID NO: ______) followed by the linker sequence GGGSSGGGSG (SEQ ID NO: ______) followed by IgG4 CH3 sequence GQPREPQVYTLPPSQEEMTKNQVSLTCLVKGFYPSDIAVEWESNGQPENNYKTTPPVLD SDGSFFLYSRLTVDKSRWQEGNVFSCSVMHEALHNHYTQKSLSLSLGK (SEQ ID NO: ______).

Thus, the entire linker/spacer region can comprise the sequence: ESKYGPPCPPCPGGGSSGGGSGGQPREPQVYTLPPSQEEMTKNQVSLTCLVKGFYPSDIA VEWESNGQPENNYKTTPPVLDSDGSFFLYSRLTVDKSRWQEGNVFSCSVMHEALHNHY TQKSLSLSLGK (SEQ ID NO: ______). In some cases, the spacer has 1, 2, 3, 4, or 5 single amino acid changes (e.g., conservative changes) compared to SEQ ID NO: ______. In some cases, the IgG4 Fc hinge/linker region that is mutated at two positions (L235E; N297Q) in a manner that reduces binding by Fc receptors (FcRs).

### Transmembrane Domain

A variety of transmembrane domains can be used in the. Table 2 includes examples of suitable transmembrane domains. Where a spacer region is present, the transmembrane domain is located carboxy terminal to the spacer region.

TABLE 2
Examples of Transmembrane Domains

| Name | Accession | Length | Sequence |
|---|---|---|---|
| CD3z | J04132.1 | 21 aa | LCYLLDGILFIYGVILTALFL (SEQ ID NO: _) |
| CD28 | NM_006139 | 27 aa | FWVLVVVGGVLACYSLLVTVAFIIFWV (SEQ ID NO: _) |
| CD28(M) | NM_006139 | 28 aa | MFWVLVVVGGVLACYSLLVTVAFIIFWV (SEQ ID NO: _) |
| CD4 | M35160 | 22 aa | MALIVLGGVAGLLLFIGLGIFF (SEQ ID NO: _) |
| CD8tm | NM_001768 | 21 aa | IYIWAPLAGTCGVLLLSLVIT (SEQ ID NO: _) |
| CD8tm2 | NM_001768 | 23 aa | IYIWAPLAGTCGVLLLSLVITLY (SEQ ID NO: _) |
| CD8tm3 | NM_001768 | 24 aa | IYIWAPLAGTCGVLLLSLVITLYC (SEQ ID NO: _) |
| 41BB | NM_001561 | 27 aa | IISFFLALTSTALLFLLFFLTLRFSVV (SEQ ID NO: _) |

### Costimulatory Domain

The costimulatory domain can be any domain that is suitable for use with a CD3ζ signaling domain. In some cases, the costimulatory domain is a CD28 costimulatory domain that includes a sequence that is at least 90%, at least 95%, at least 98% identical to or identical to: RSKRSRGGHSDYMNMTPRRPGPTRKHYQPYAPPRDFAAYRS (SEQ ID NO:23; LL to GG amino acid change double underlined). In some cases, the CD28 co-signaling domain has 1, 2, 3, 4 of 5 amino acid changes (preferably conservative and preferably not in the underlined GG sequence) compared to SEQ ID NO: ______. In some cases the co-signaling domain is a 4-1BB co-signaling domain that includes a sequence that is at least 90%, at least 95%, at least 98% identical to or identical to: KRGRKKLLYIFKQPFMRPVQTTQEEDGCSCRFPEEEEGGCEL (SEQ ID NO:24). In some cases, the 4-1BB co-signaling domain has 1, 2, 3, 4 of 5 amino acid changes (preferably conservative) compared to SEQ ID NO: ______.

The costimulatory domain(s) are located between the transmembrane domain and the CD3ζ signaling domain. Table 3 includes examples of suitable costimulatory domains together with the sequence of the CD3ζ signaling domain.

TABLE 3
CD3ζ Domain and Examples of Costimulatory Domains

| Name | Accession | Length | Sequence |
|---|---|---|---|
| CD3ζ | J04132.1 | 113 aa | RVKFSRSADAPAYQQGQNQLYNELNLGRREEYDVLDKRRGR |
| | | | DPEMGGKPRRKNPQEGLYNELQKDKMAEAYSEIGMKGERR |
| | | | RGKGHDGLYQGLSTATKDTYDALHMQALPPR |
| | | | (SEQ ID NO: _) |
| CD28 | NM_006139 | 42 aa | RSKRSRLLHSDYMNMTPRRPGPTRKHYQPYAPPRDFAAYRS |
| | | | (SEQ ID NO: _) |
| CD28gg* | NM_006139 | 42 aa | RSKRSRGGHSDYMNMTPRRPGPTRKHYQPYAPPRDFAAYRS |
| | | | (SEQ ID NO: _) |
| 41BB | NM_001561 | 42 aa | KRGRKKLLYIFKQPFMRPVQTTQEEDGCSCRFPEEEEGGCEL |
| | | | (SEQ ID NO: _) |
| OX40 | | 42 aa | ALYLLRRDQRLPPDAHKPPGGGSFRTPIQEEQADAHSTLAKI |
| | | | (SEQ ID NO: _) |

In various embodiments: the costimulatory domain is selected from the group consisting of: a costimulatory domain depicted in Table 3 or a variant thereof having 1-5 (e.g., 1 or 2) amino acid modifications, a CD28 costimulatory domain or a variant thereof having 1-5 (e.g., 1 or 2) amino acid modifications, a 4-1BB costimulatory domain or a variant thereof having 1-5 (e.g., 1 or 2) amino acid modifications and an OX40 costimulatory domain or a variant thereof having 1-5 (e.g., 1 or 2) amino acid modifications. In certain embodiments, a 4-1BB costimulatory domain or a variant thereof having 1-5 (e.g., 1 or 2) amino acid modifications in present. In some embodiments there are two costimulatory domains, for example a CD28 co-stimulatory domain or a variant thereof having 1-5 (e.g., 1 or 2) amino acid modifications (e.g., substitutions) and a 4-1BB co-stimulatory domain or a variant thereof having 1-5 (e.g., 1 or 2) amino acid modifications (e.g., substitutions). In various embodiments the 1-5 (e.g., 1 or 2) amino acid modification are substitutions. The costimulatory domain is amino terminal to the CD3ζ signaling domain and in some cases a short linker consisting of 2-10, e.g., 3 amino acids (e.g., GGG) is positioned between the costimulatory domain and the CD3ζ signaling domain.

### CD3ζ Signaling Domain

The CD3ζ Signaling domain can be any domain that is suitable for use with a CD3ζ signaling domain. In some cases, the CD3ζ signaling domain includes a sequence that is at least 90%, at least 95%, at least 98% identical to or identical to: RVKFSRSADAPAYQQGQNQLYNELNLGRREEYDVLDKRRGRDPEMGGKPRRKNPQEG LYNELQKDKMAEAYSEIGMKGERRRGKGHDGLYQGLSTATKDTYDALHMQALPPR ((SEQ ID NO: ______)). In some cases, the CD3 signaling has 1, 2, 3, 4 of 5 amino acid changes (preferably conservative) compared to SEQ ID NO:21.

### Pharmaceutical Compositions

In an aspect is provided a pharmaceutical composition including a therapeutically effective amount of a recombinant protein as described herein, including embodiments thereof, and a pharmaceutically acceptable excipient.

### Nucleic Acid Compositions

In an aspect, an isolated nucleic acid encoding a recombinant protein as described herein, including embodiments thereof is provided.

### Methods of Treatment

The compositions provided herein, including embodiments thereof, are contemplated as providing effective treatments for diseases such as cancer (e.g., mantel cell lymphoma). Thus, in an aspect is provided a method of treating cancer in a subject in need thereof, the method including administering to a subject a therapeutically effective amount of a recombinant protein as provided herein, including embodiments thereof, thereby treating cancer in the subject.

In embodiments, cancer is lymphoma, leukemia, or myeloma. In embodiments, cancer is lymphoma. In embodiments, cancer is leukemia. In embodiments, cancer is myeloma.

In embodiments, the lymphoma is non-Hodgkin's lymphoma. In embodiments, the non-Hodgkin's lymphoma is mantle cell lymphoma, follicular lymphoma, diffuse large B-cell lymphoma, marginal zone lymphoma or Burkitt's lymphoma. In embodiments, the non-Hodgkin's lymphoma is mantle cell lymphoma. In embodiments, the non-Hodgkin's lymphoma is follicular lymphoma. In embodiments, the non-Hodgkin's lymphoma is diffuse large B-cell lymphoma. In embodiments, the non-Hodgkin's lymphoma is marginal zone lymphoma. In embodiments, the non-Hodgkin's lymphoma is Burkitt's lymphoma.

In embodiments, the leukemia is lymphoblastic leukemia, chronic lymphocytic leukemia or hairy cell leukemia. In embodiments, the leukemia is lymphoblastic leukemia. In embodiments, the leukemia is chronic lymphocytic leukemia. In embodiments, the leukemia is hairy cell leukemia.

The recombinant proteins provided herein including embodiments thereof, may be administered in combination with additional therapeutic agents. Thus, in embodiments, the method provided herein, including embodiments thereof, further includes administering to the subject a second therapeutic agent.

### Method of Inhibiting Proliferation of a Cell

The recombinant protein compositions provided herein, including embodiments thereof, are useful for inducing cytotoxicity in a cell (e.g., cancer cell), thereby promoting cell death. Thus, in an aspect is provided a method of inhibiting proliferation of a cell, the method including: (i) contacting a cell with a recombinant protein as provided herein, including embodiments thereof, thereby forming a contacted cell; and (ii) allowing the recombinant protein as provided herein, including embodiments thereof, to bind to the contacted cell, thereby inhibiting proliferation of the cell.

In embodiments, the cell is a cancer cell. In embodiments, the cancer cell is a lymphoma, leukemia, or myeloma cancer cell. In embodiments, the cancer cell is a lymphoma cancer cell. In embodiments, the cancer cell is a leukemia cancer. In embodiments, the cancer cell is a myeloma cancer cell.

In embodiments, the cancer cell is a non-Hodgkin's lymphoma cancer cell. In embodiments, the non-Hodgkin's lymphoma cancer cell is a mantle cell lymphoma (MCL), follicular lymphoma, diffuse large B-cell lymphoma, marginal zone lymphoma, or Burkitt's lymphoma cancer cell. In embodiments, the non-Hodgkin's lymphoma cancer cell is a mantle cell lymphoma (MCL) cancer cell. In embodiments, the non-Hodgkin's lymphoma cancer cell is a follicular lymphoma cancer cell. In embodiments, the non-Hodgkin's lymphoma cancer cell is a diffuse large B-cell lymphoma cancer cell. In embodiments, the non-Hodgkin's lymphoma cancer cell is a marginal zone lymphoma cancer cell. In embodiments, the non-Hodgkin's lymphoma cancer cell is a Burkitt's lymphoma cancer cell.

In embodiments, the cancer cell is a leukemia cancer cell. In embodiments, the leukemia cancer cell is a lymphoblastic leukemia, chronic lymphocytic leukemia, or hairy cell leukemia cancer cell. In embodiments, the leukemia cancer cell is a lymphoblastic leukemia cancer cell. In embodiments, the leukemia cancer cell is a chronic lymphocytic leukemia cancer cell. In embodiments, the leukemia cancer cell is a hairy cell leukemia cancer cell.

### EXAMPLES

The following examples are intended to further illustrate certain embodiments of the disclosure. The examples are put forth so as to provide one of ordinary skill in the art and are not intended to limit its scope.

### Example 1. Mantle Cell Lymphoma-Specific Immunotherapeutics: Chimeric Antigen Receptor (CAR) T Cell Therapy and Bispecific T-Cell Engaging Monoclonal Antibody

Immunotherapeutics are currently developed against a range of targets and employ unique mechanisms of action to combat tumors. However, all pursue cell surface proteins that are simultaneously present on malignant cells and their benign counterparts. Consequently, current antibody-based antitumor therapeutics will inevitably deplete healthy, non-malignant cells in the process of eliminating tumors. Chimeric antigen receptor T-cell (CART) therapies are a prime example where ubiquitous, tumor associated antigens are targeted causing adverse effects such as lymphocytopenia. Equally important to specify is the origin of the parent antibody. Many immunotherapeutics are derived from murine generated antibodies, which could elicit immune responses against these components and potentially abrogate the efficacy of the therapy.

Here, we describe a mantle cell lymphoma (MCL)-specific, human immunoglobulin light chain variable fragment (VL) derived from human antibody phage display library. The VL was developed by screening a MCL line by subtractive and subsequent competitive biopanning. The result was a light chain only antibody, termed LC-Ab, with high specificity to MCL lines and patient samples, but not normal, healthy lymphocytes.

We subsequently cloned the MCL-specific VL onto a second generation CAR backbone containing a CD8a hinge, transmembrane domain, a 4-1BB co-stimulatory domain, and a CD3ζ activation domain to develop a novel CAR. The engineered MCL-specific CAR-Ts secreted high quantities of TNF-α and IFN-γ in response to MCL lines including JeKo-1, Mino and Z-138 but did not respond to normal B cells. Additionally, the CAR-Ts demonstrated potent cytotoxicity specifically against MCL lines but not normal B cells at different effector to target ratios. In vitro cytotoxicity specifically against MCLs was confirmed with a panel of MCL lines including JeKo-1, Mino, Z-138, REC-1, MAVER-1 and NCEB-1 compared to no reactivity against several non-MCL lines including RL (follicular lymphoma), RS4;11 (acute lymphoblastic leukemia), and Raji (Burkett lymphoma). These primary results will be validated in vivo in the ongoing study.

In addition to CAR T cells, we investigated integrating our MCL-specific VL onto a bi-specific T-cell engaging monoclonal antibody (BiTe). The MCL-specific antibody VL was paired with a scFv of a CD3 antibody. Our in vitro data demonstrated the ability of our bispecific antibody to recruit T cells and induce cytotoxicity against MCL line JeKo-1 but no effects against normal B cells at different effector to target ratios. The antibody exhibited potent anti-tumor effects against the aforementioned panel of MCL lines but had no reactivity against non-MCL panel. Further studies to confirm MCL-specific cytotoxicity in vivo is planned and ongoing.

### Example 2. CAR Construct

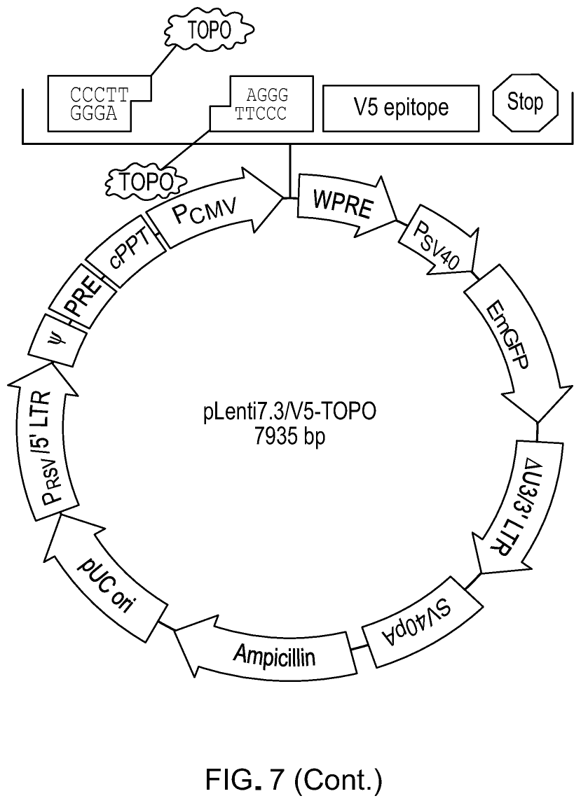

The light chain variable domain (VL) with specificity for mantle cell lymphoma linked with CD8a hinge and transmembrane domains, CD137 (a co-stimulatory receptor in T cells [4-1BB]) and CD3-zeta (a signal-transduction component of the T-cell antigen receptor) signaling domains was synthesized and cloned into pLenti7.3/V5-TOPO lentivirus vector (FIG. 7). The sequence of CD8a hinge and transmembrane domains, 4-1BB and CD3-zeta signaling domains were as shown in FIG. 7. Schematic of the vector shows major functional elements: 3′ LTR-3′ long terminal repeat; 5′ LTR-5′ long terminal repeat; Amp R—ampicillin resistance gene; cPPT—central polypurine tract with central termination sequence; RRE—rev response element; VL—the light chain variable domain; TM—transmembrane; WPRE—wood chuck hepatitis virus post-transcriptional regulatory element (FIG. 7).

### Example 3. Phage Display

Jeko cells were stained with anti-CD20-APC and anti-BAFFR-PE antibodies (BD Biosciences). CD20 and BAFFR labeled Jeko cells were mixed with PBMC followed by incubation with 2×10¹² ScFv phage display library for 1 hour at 4° C. Labeled Jeko cells were sorted out and their bound phages were eluted, titered and amplified for the next round of biopanning. After the 4th round of biopanning, predominant antibody binding domains were identified by PCR analysis of 48 individual phages eluted from cells.

### Example 4. Phage Binding Assay

Phage clones with predominant antibody binding domain sequences were produced and used for staining of different tumor cells. Phages were incubated with tumor cells for 1 hour at 4° C. followed by staining with anti-M13 phage-FITC antibody (Fitzgerald).

### Example 5. CART CTL Assays

Target cells (human tumor cell lines or normal B cells) were labeled with chromium-51 (51Cr, Perkin Elmer, Waltham, Mass.) for 51Cr release assay. Engineered chimeric antigen receptor T cells were added to labeled target cells and incubated up to 4 hours. 51Cr released into the supernatant was detected with a Wizard Automatic Gamma Counter (Perkin Elmer).

### Example 6. Cytokine-Release Assays

Engineered chimeric antigen receptor T cells were incubated with different human tumor cell lines at 10:1 ratio for 24 hours. Cytokine IFN-gamma and TNFα released into the supernatant were measured by ELISA with anti-IFN-gamma and anti-TNFα antibodies.

### Example 7. BiTe Cytotoxicity Assays

Target cells (human tumor lines or normal B cells) were labeled with chromium-51 (51Cr, Perkin Elmer, Waltham, Mass.) for 51Cr release assay. Antibodies and purified T cells were added to labeled target cells and incubated up to 4 hours. T cells were enriched from PBMC (T cell enrichment kit, Stemcell Technologies, Vancouver, Canada). 51Cr released into the supernatant was detected with a Wizard Automatic Gamma Counter (Perkin Elmer).

### Example 8. Tumor-Specific CAR T-Cell Cytokine Release and Degranulation

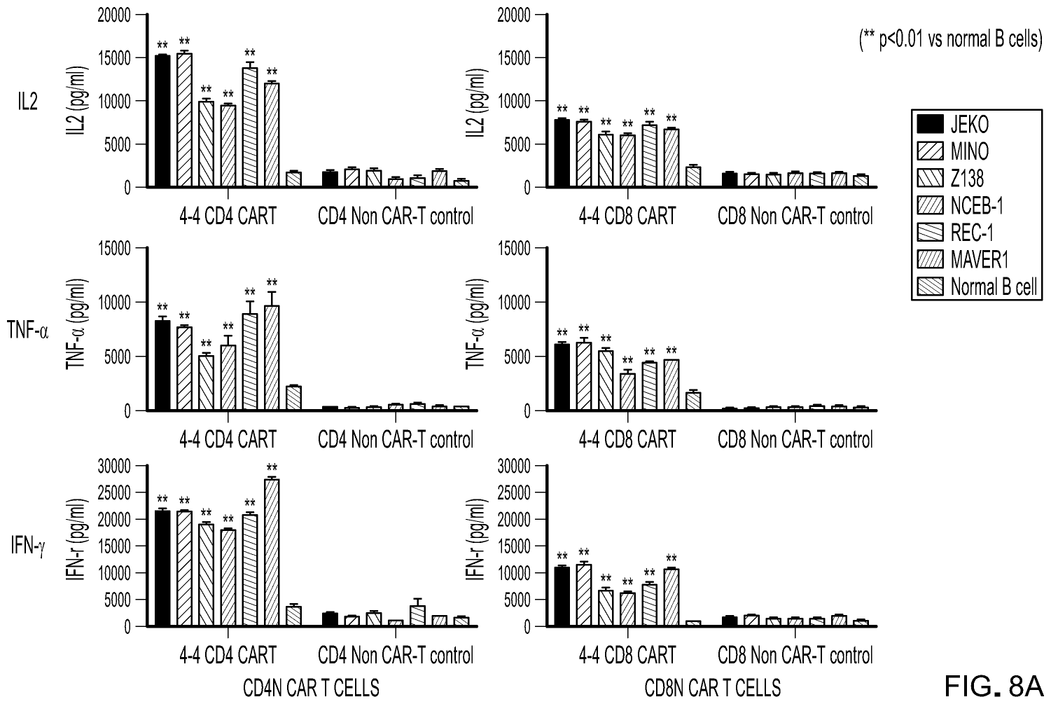

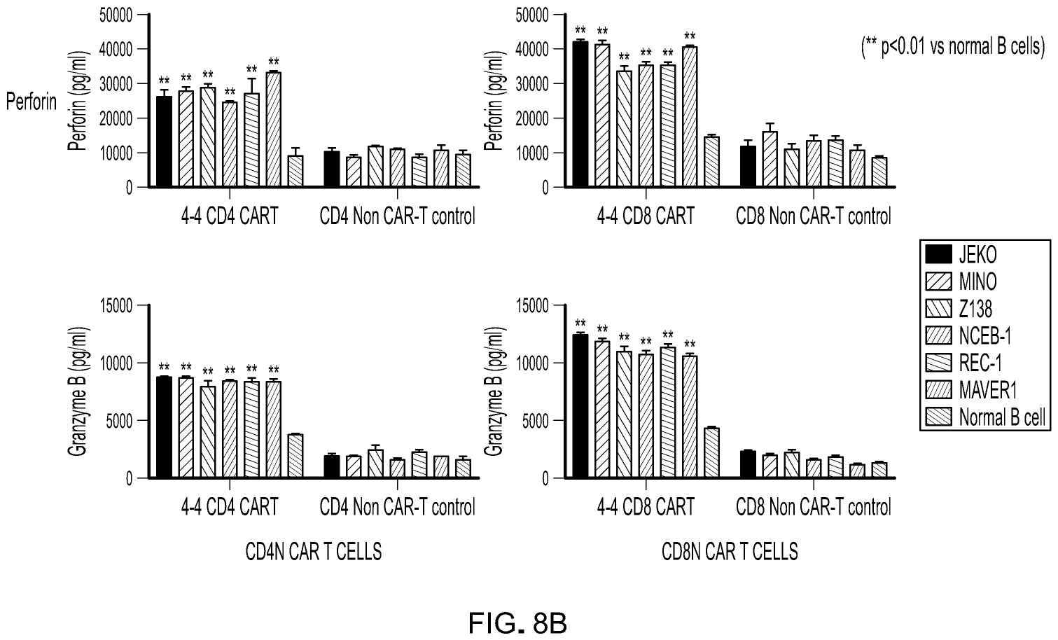

ELISA measurements of (FIG. 8A) cytokines: IL2, TNF-α, and INF-γ; or (FIG. 8B) granules: perform and granzyme; from supernatant of MCL (4-4) CD4 or CD8 CAR T cells incubated with various tumor lines or normal B cells. The CAR was active against tumor cells.

### Example 9. In Vivo MCL (4-4) CAR T-Cell Treatment

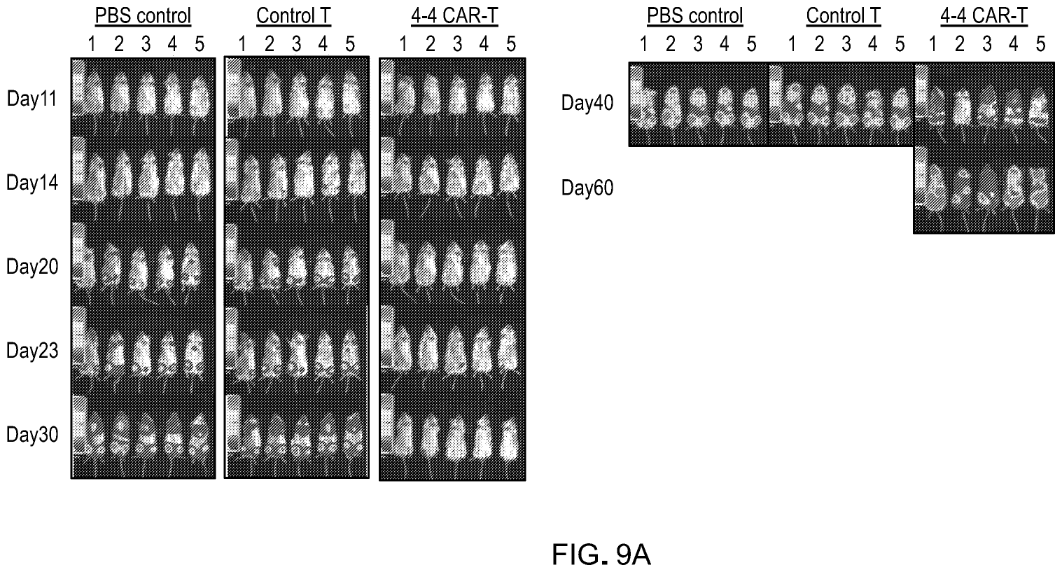

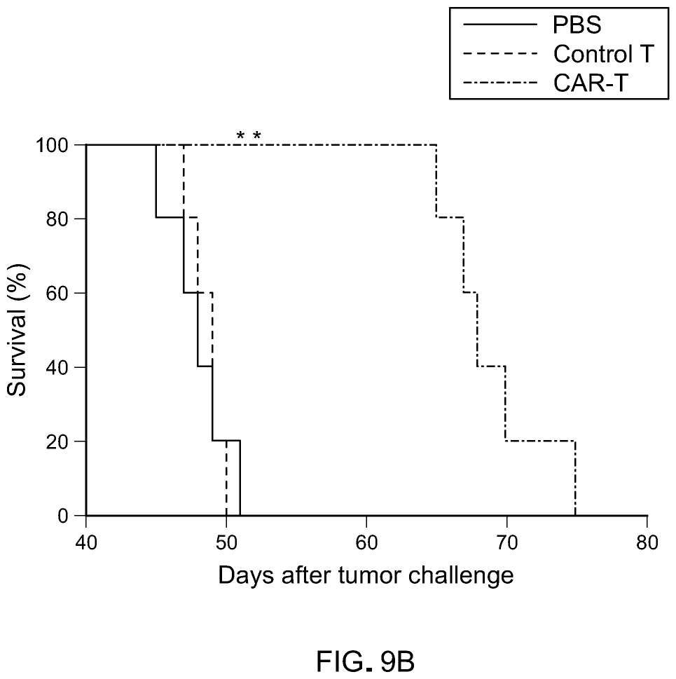

FIGS. 9A and 9B depict the results of a study in NSG mice (n=5) following intravenous (IV) tumor challenge (5×104 cells/mouse) on day 0 with luciferase-expressing Z-138. Activated CAR T-cell treatments were infused by IV on day 11. Treatments consisted of 2.5×106 CD4+TN+1×106 CD8+TN CAR-T cells. Non-transduced (control) T cells (3.5×106 cells) from the same donor or PBS was used as controls.

### Example 10. In Vivo MCL (4-4) BiTE Antibody Treatment

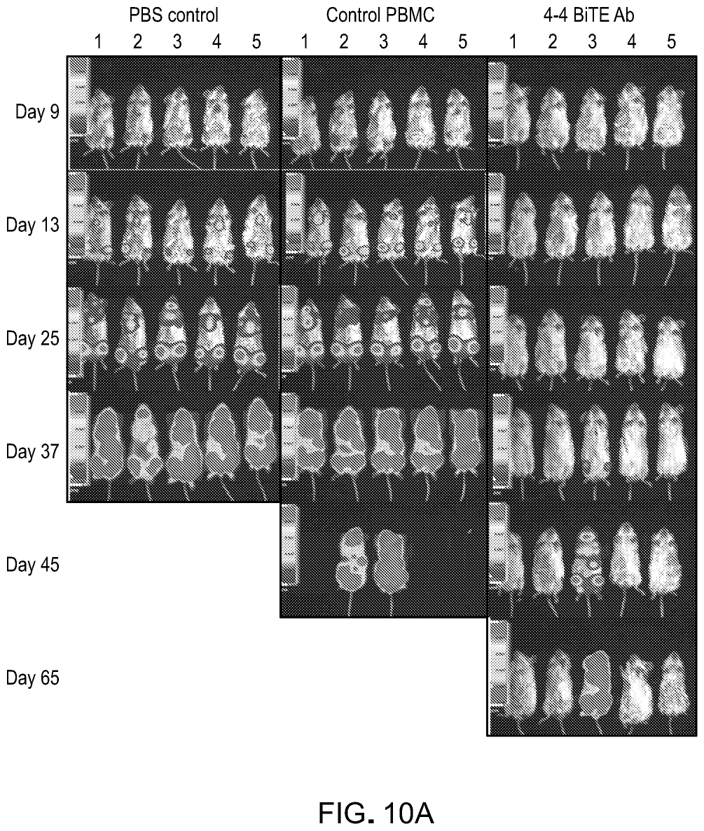

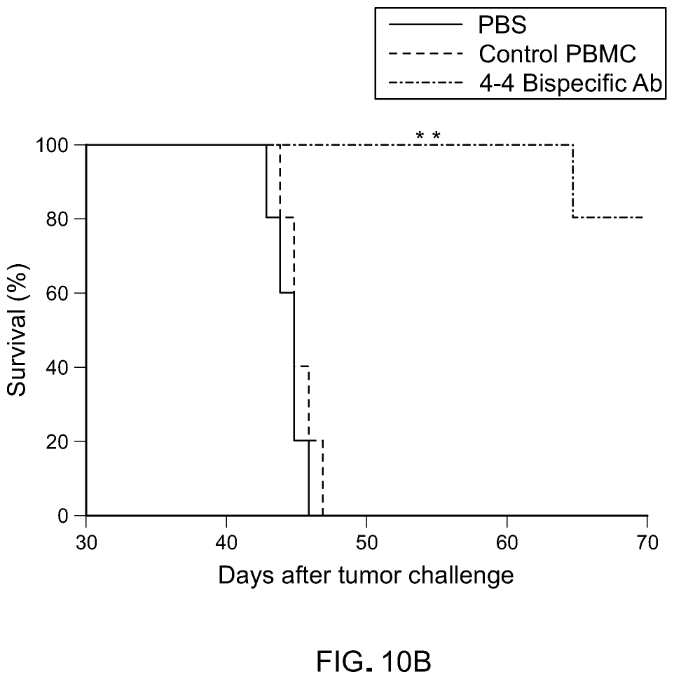

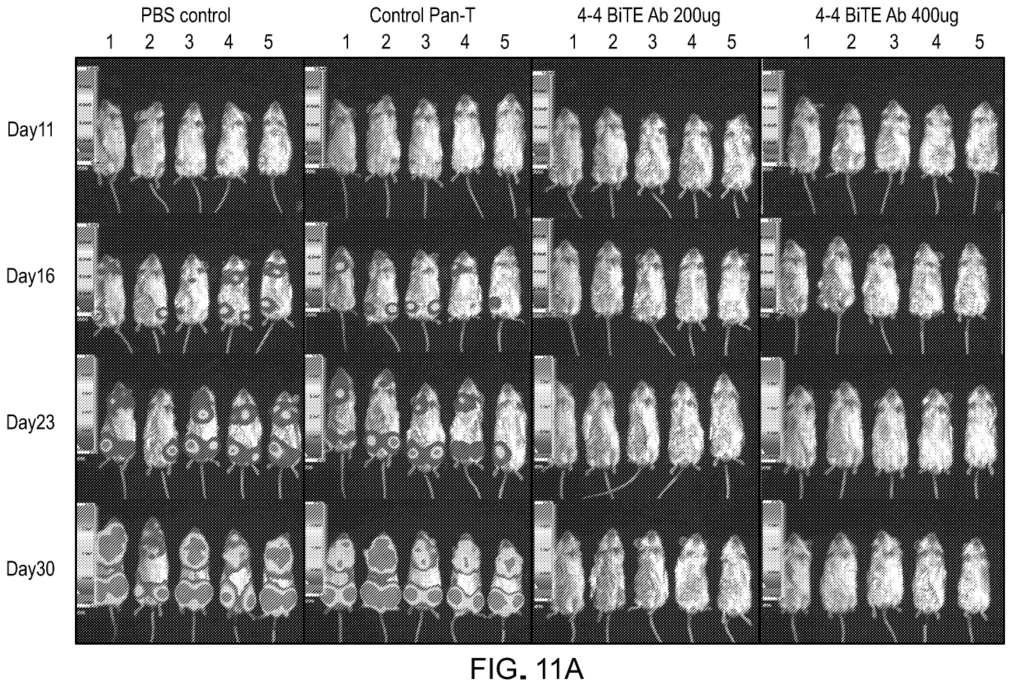

FIGS. 10A and 10B depict the results of a study in (NSG mice (n=5) following intravenous (IV) tumor challenge (5×104 cells/mouse) on day 0 with luciferase-expressing Z-138. Three treatments given on days 4, 8, and 12 consisted of 400 μg MCL (4-4) antibody+10×106 isolated PBMC. PBMC from the same donor alone or PBS were used as controls.

### Example 11. In Vivo MCL (4-4) BiTE Antibody Treatment

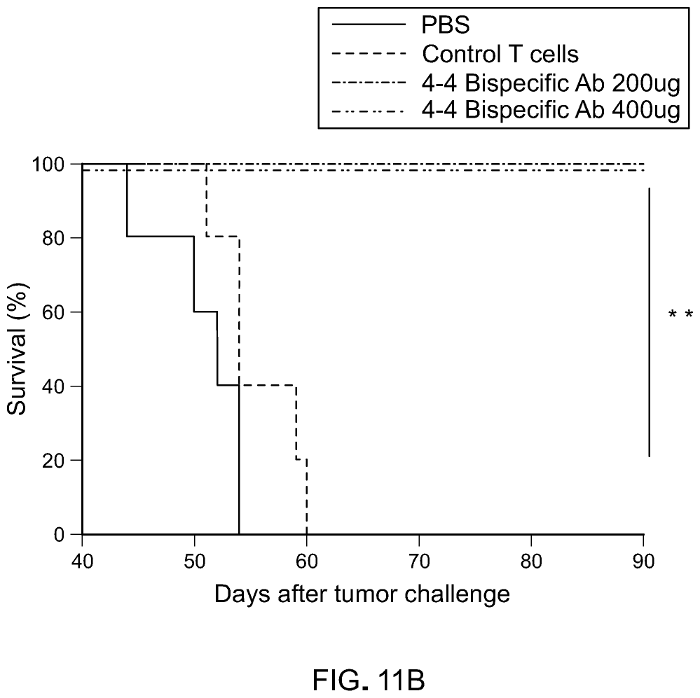

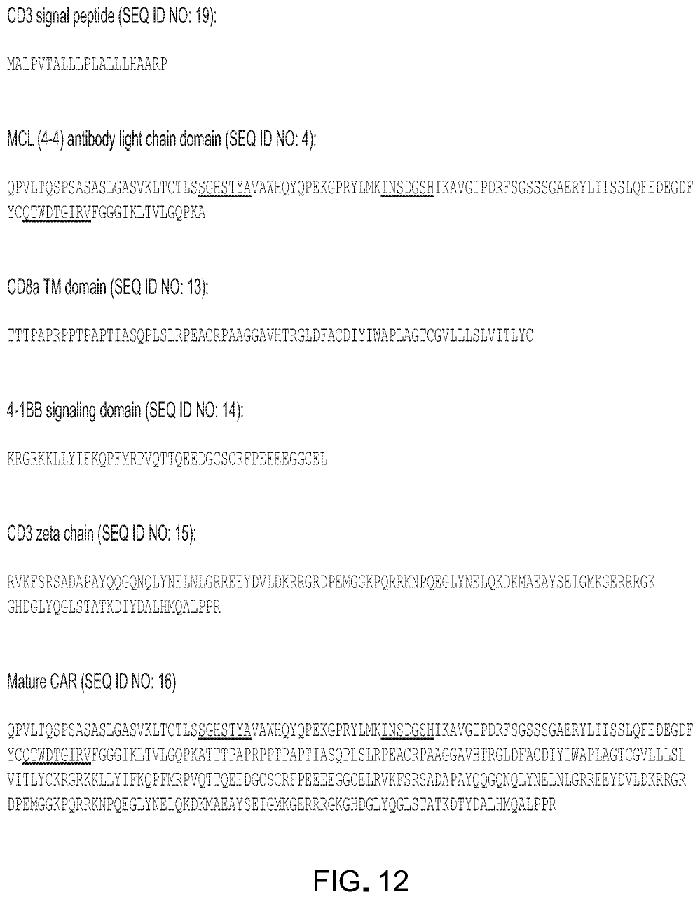

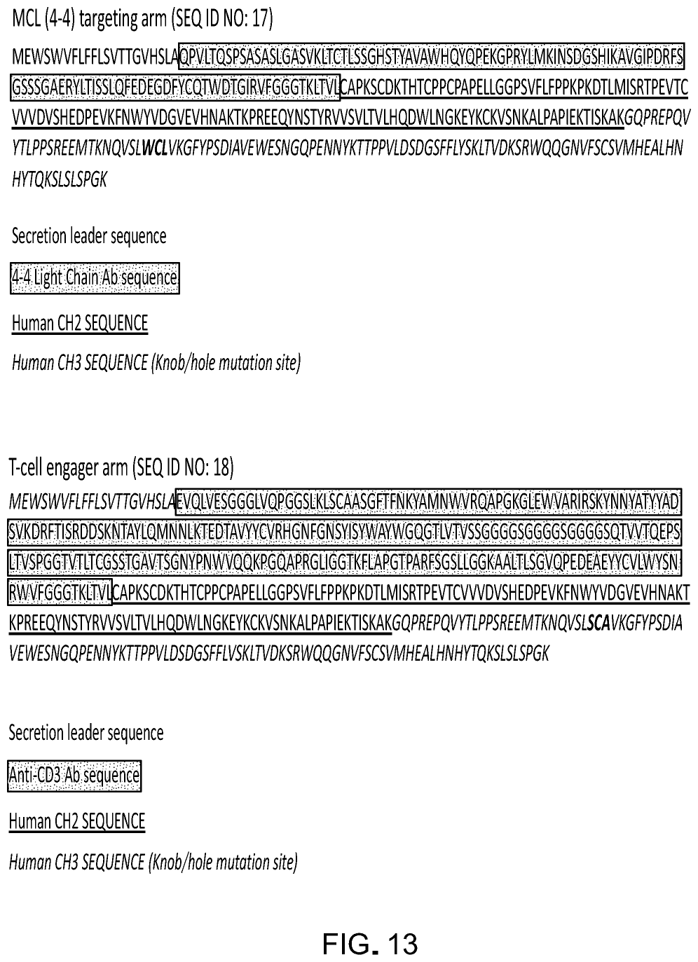

FIGS. 11A-11B depict the results of a study in of NSG mice (n=5) following intravenous (IV) tumor challenge (5×104 cells/mouse) on day 0 with luciferase-expressing Z-138. Four treatments given on days 11, 14, 17, and 20 consisted of 200 μg or 400 μg MCL (4-4) antibody+5×106 isolated pan T cells. T cells from the same donor alone or PBS were used as controls.

### INFORMAL SEQUENCE LISTING

(CDR L1)
SEQ ID NO: 1
SGHSTYA
(CDR L2)
SEQ ID NO: 2
INSDGSH
(CDR L3)
SEQ ID NO: 3
QTWDTGIRV
(4-4 Light Chain Variable Region Protein Sequence (CDRs in bold))
SEQ ID NO: 4
QPVLTQSPSASASLGASVKLTCTLSSGHSTYAVAWHQYQPEKGPRYLMKINSDGSHIK
AVGIPDRFSGSSSGAERYLTISSLQFEDEGDFYCQTWDTGIRVFGGGTKLTVLGQPKA
(4-4 Light Chain Variable Region DNA Sequence)
SEQ ID NO: 5
CAGCCTGTGCTGACTCAATCGCCCTCTGCCTCTGCCTCCCTGGGAGCCTCGGTCAAG
CTCACCTGCACTCTGAGCAGTGGCCACAGTACATACGCCGTCGCATGGCATCAATAC
CAGCCAGAGAAGGGCCCTCGATATTTGATGAAGATTAACAGTGATGGCAGCCACAT
CAAGGCGGTCGGGATTCCTGATCGATTCTCAGGCTCCAGCTCTGGGGCTGAGCGCTA
CCTCACCATCTCCAGCCTCCAATTTGAGGATGAGGGTGACTTTTATTGTCAGACGTG
GGACACTGGCATTCGAGTGTTCGGCGGAGGGACCAAATTGACCGTCCTCGGTCAGC
CCAAGGCT
(DNA sequence for CD8a hinge and transmembrane domains)
SEQ ID NO: 6
ACCACGACGCCAGCGCCGCGACCACCAACACCGGCGCCCACCATCGCGTCGCAGCC
CCTGTCCCTGCGCCCAGAGGCGTGCCGGCCAGCGGCGGGGGGCGCAGTGCACACGA
GGGGGCTGGACTTCGCCTGTGATATCTACATCTGGGCGCCCTTGGCCGGGACTTGTG
GGGTCCTTCTCCTGTCACTGGTTATCACCCTTTACTGC
(DNA sequence for 4-1BB signaling domain)
SEQ ID NO: 7
AAACGGGGCAGAAAGAAACTCCTGTATATATTCAAACAACCATTTATGAGACCAGT
ACAAACTACTCAAGAGGAAGATGGCTGTAGCTGCCGATTTCCAGAAGAAGAAGAAG
GAGGATGTGAACTG
(DNA sequence for CD3-zeta signaling domain)
SEQ ID NO: 8
AGAGTGAAGTTCAGCAGGAGCGCAGACGCCCCCGCGTACCAGCAGGGCCAGAACC
AGCTCTATAACGAGCTCAATCTAGGACGAAGAGAGGAGTACGATGTTTTGGACAAG
AGACGTGGCCGGGACCCTGAGATGGGGGGAAAGCCGAGAAGGAAGAACCCTCAGG
AAGGCCTGTACAATGAACTGCAGAAAGATAAGATGGCGGAGGCCTACAGTGAGATT
GGGATGAAAGGCGAGCGCCGGAGGGGCAAGGGGCACGATGGCCTTTACCAGGGTC
TCAGTACAGCCACCAAGGACACCTACGACGCCCTTCACATGCAGGCCCTGCCCCCTC
GCTAA
(DNA sequences for CD8a hinge and transmembrane domains,
4-1BB, and CD3-zeta signaling domains)
SEQ ID NO: 9
ACCACGACGCCAGCGCCGCGACCACCAACACCGGCGCCCACCATCGCGTCGCAGCC
CCTGTCCCTGCGCCCAGAGGCGTGCCGGCCAGCGGCGGGGGGCGCAGTGCACACGA
GGGGGCTGGACTTCGCCTGTGATATCTACATCTGGGCGCCCTTGGCCGGGACTTGTG
GGGTCCTTCTCCTGTCACTGGTTATCACCCTTTACTGCAAACGGGGCAGAAAGAAAC
TCCTGTATATATTCAAACAACCATTTATGAGACCAGTACAAACTACTCAAGAGGAAG
ATGGCTGTAGCTGCCGATTTCCAGAAGAAGAAGAAGGAGGATGTGAACTGAGAGTG
AAGTTCAGCAGGAGCGCAGACGCCCCCGCGTACCAGCAGGGCCAGAACCAGCTCTA
TAACGAGCTCAATCTAGGACGAAGAGAGGAGTACGATGTTTTGGACAAGAGACGTG
GCCGGGACCCTGAGATGGGGGGAAAGCCGAGAAGGAAGAACCCTCAGGAAGGCCT
GTACAATGAACTGCAGAAAGATAAGATGGCGGAGGCCTACAGTGAGATTGGGATGA
AAGGCGAGCGCCGGAGGGGCAAGGGGCACGATGGCCTTTACCAGGGTCTCAGTACA
GCCACCAAGGACACCTACGACGCCCTTCACATGCAGGCCCTGCCCCCTCGCTAA
(scFv targeting CD3 (DNA))
SEQ ID NO: 10
GAGGTGCAGCTGGTGGAAAGCGGAGGAGGCCTGGTGCAGCCCGGAGGCTCCCTGAA
GCTGAGCTGTGCCGCCAGCGGCTTCACCTTCAACAAGTACGCCATGAACTGGGTGA
GACAGGCCCCTGGCAAAGGCCTGGAGTGGGTGGCCAGGATCAGGTCCAAGTACAAC
AATTACGCCACCTATTACGCCGACAGCGTCAAGGACAGGTTCACCATCAGCAGGGA
CGACAGCAAGAATACCGCTTACCTGCAGATGAACAACCTGAAGACCGAGGACACCG
CCGTGTATTACTGCGTGAGGCACGGCAACTTCGGCAACAGCTACATCTCCTACTGGG
CCTATTGGGGCCAGGGCACCCTGGTCACAGTGAGCAGCGGAGGAGGAGGCAGCGG
AGGAGGAGGAAGCGGAGGCGGAGGATCCCAGACCGTGGTGACCCAGGAGCCTAGC
CTGACAGTGTCCCCTGGCGGCACCGTGACCCTGACATGTGGCTCCAGCACAGGCGCC
GTGACAAGCGGCAACTACCCCAACTGGGTGCAGCAGAAGCCTGGCCAGGCTCCTAG
GGGACTGATCGGCGGCACCAAGTTCCTGGCTCCCGGCACACCTGCCAGATTCAGCG
GCAGCCTGCTGGGAGGAAAGGCCGCTCTGACCCTGAGCGGCGTGCAGCCCGAAGAT
GAGGCCGAGTACTATTGCGTGCTGTGGTACAGCAACAGATGGGTGTTCGGCGGCGG
CACCAAGCTCACCGTGCTG
(CH2 and CH3 Sequence for 4-4)
SEQ ID NO: 11
CAPKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPA
PIEKTISKAKGQPREPQVYTLPPSREEMTKNQVSLWCLVKGFYPSDIAVEWESNGQPEN
NYKTTPPVLDSDGSFFLYSKLTVDKSRWQQGNVFSCSVMHEALHNHYTQKSLSLSPGK
(CH2 and CH3 Sequence for CD3)
SEQ ID NO: 12
CAPKSCDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPA
PIEKTISKAKGQPREPQVYTLPPSREEMTKNQVSLSCAVKGFYPSDIAVEWESNGQPENN
YKTTPPVLDSDGSFFLVSKLTVDKSRWQQGNVFSCSVMHEALHNHYTQKSLSLSPGK

1. A recombinant protein comprising:
(i) an antibody region comprising a light chain variable domain comprising a CDR L1 as set forth in SEQ ID NO:1, a CDR L2 as set forth in SEQ ID NO:2, and a CDR L3 as set forth in SEQ ID NO:3; and
(ii) a transmembrane domain.

2. The recombinant protein of claim 1, wherein said light chain variable domain comprises the sequence of SEQ ID NO:4.

3. The recombinant protein of claim 1, wherein said transmembrane domain is a CD8 transmembrane domain, a CD28 transmembrane domain, a CD4 transmembrane domain or a CD3ζ transmembrane domain.

4.-5. (canceled)

6. The recombinant protein of claim 1, further comprising an intracellular co-stimulatory signaling domain and a CD3ζ intracellular T-cell signaling domain.

7. The recombinant protein of claim 6, wherein said intracellular co-stimulatory signaling domain is a 4-1BB intracellular co-stimulatory signaling domain, a CD28 intracellular co-stimulatory signaling domain, a ICOS intracellular co-stimulatory signaling domain, or an OX-40 intracellular co-stimulatory signaling domain.

8.-24. (canceled)

25. An isolated nucleic acid encoding a recombinant protein of claim 1.

26. (canceled)

27. A method of treating cancer in a subject in need thereof, said method comprising administering to a subject a therapeutically effective amount of a recombinant protein of claim 1, thereby treating cancer in said subject.

28. A recombinant protein comprising:
(i) a first antibody region capable of binding an effector cell ligand; and
(ii) a second antibody region, comprising a light chain variable domain comprising a CDR L1 as set forth in SEQ ID NO:1, a CDR L2 as set forth in SEQ ID NO:2 and a CDR L3 as set forth in SEQ ID NO:3.

29. The recombinant protein of claim 28, wherein said effector cell ligand is a CD3 protein.

30. The recombinant protein of claim 28, wherein said light chain variable domain comprises the sequence of SEQ ID NO:4.

31.-33. (canceled)

34. The recombinant protein of claim 28, further comprising a first constant heavy chain 3 (CH3) domain bound to said first antibody region through a first constant heavy chain 2 (CH2) domain, and a second constant heavy chain 3 (CH3) domain bound to said second antibody region through a second constant heavy chain 2 (CH2) domain.

35. The recombinant protein of claim 34, wherein said first CH3 domain is bound to said second CH3 domain.

36.-42. (canceled)

43. A method of treating cancer in a subject in need thereof, said method comprising administering to a subject a therapeutically effective amount of a recombinant protein of claim 28, thereby treating cancer in said subject.

44.-55. (canceled)


SEQUENCE LISTING (46 sequences)

SEQ ID NO: 1 - protein, 7 aa, Artificial Sequence
  Synthetic peptide
SGHSTYA

SEQ ID NO: 2 - protein, 7 aa, Artificial Sequence
  Synthetic peptide
INSDGSH

SEQ ID NO: 3 - protein, 9 aa, Artificial Sequence
  Synthetic peptide
QTWDTGIRV

SEQ ID NO: 4 - protein, 116 aa, Artificial Sequence
  Synthetic polypeptide
QPVLTQSPSASASLGASVKLTCTLSSGHSTYAVAWHQYQPEKGPRYLMKINSDGSHIKAVGIPDRFSGSSSGAERYLTISSLQFEDEGDFYCQTWDTGIRVFGGGTKLTVLGQPKA

SEQ ID NO: 5 - DNA, 348 bp, Artificial Sequence
  Synthetic polypeptide
cagcctgtgctgactcaatcgccctctgcctctgcctccctgggagcctcggtcaagctcacctgcactctgagcagtggccacagtacatacgccgtcgcatggcatcaataccagccagagaagggccctcgatatttgatgaagattaacagtgatggcagccacatcaaggcggtcgggattcctgatcgattctcaggctccagctctggggctgagcgctacctcaccatctccagcctccaatttgaggatgagggtgacttttattgtcagacgtgggacactggcattcgagtgttcggcggagggaccaaattgaccgtcctcggtcagcccaaggct

SEQ ID NO: 6 - DNA, 207 bp, Homo sapiens
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 7 - DNA, 126 bp, Homo sapiens
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 8 - DNA, 339 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 9 - protein, 129 aa, Artificial Sequence
  Synthetic polypeptide
ESKYGPPCPPCPGGGSSGGGSGGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - DNA, 747 bp, Artificial Sequence
  Synthetic polypeptide
gaggtgcagctggtggaaagcggaggaggcctggtgcagcccggaggctccctgaagctgagctgtgccgccagcggcttcaccttcaacaagtacgccatgaactgggtgagacaggcccctggcaaaggcctggagtgggtggccaggatcaggtccaagtacaacaattacgccacctattacgccgacagcgtcaaggacaggttcaccatcagcagggacgacagcaagaataccgcttacctgcagatgaacaacctgaagaccgaggacaccgccgtgtattactgcgtgaggcacggcaacttcggcaacagctacatctcctactgggcctattggggccagggcaccctggtcacagtgagcagcggaggaggaggcagcggaggaggaggaagcggaggcggaggatcccagaccgtggtgacccaggagcctagcctgacagtgtcccctggcggcaccgtgaccctgacatgtggctccagcacaggcgccgtgacaagcggcaactaccccaactgggtgcagcagaagcctggccaggctcctaggggactgatcggcggcaccaagttcctggctcccggcacacctgccagattcagcggcagcctgctgggaggaaaggccgctctgaccctgagcggcgtgcagcccgaagatgaggccgagtactattgcgtgctgtggtacagcaacagatgggtgttcggcggcggcaccaagctcaccgtgctg

SEQ ID NO: 11 - protein, 233 aa, Artificial Sequence
  Synthetic polypeptide
CAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 233 aa, Artificial Sequence
  Synthetic polypeptide
CAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 13 - protein, 69 aa, Homo sapiens
TTTPAPRPPTPAPTIASQPLSLRPEACRPAAGGAVHTRGLDFACDIYIWAPLAGTCGVLLLSLVITLYC

SEQ ID NO: 14 - protein, 42 aa, Homo sapiens
KRGRKKLLYIFKQPFMRPVQTTQEEDGCSCRFPEEEEGGCEL

SEQ ID NO: 15 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 16 - protein, 340 aa, Artificial Sequence
  Synthetic polypeptide
QPVLTQSPSASASLGASVKLTCTLSSGHSTYAVAWHQYQPEKGPRYLMKINSDGSHIKAVGIPDRFSGSSSGAERYLTISSLQFEDEGDFYCQTWDTGIRVFGGGTKLTVLGQPKATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 17 - protein, 365 aa, Artificial Sequence
  Synthetic polypeptide
MEWSWVFLFFLSVTTGVHSLAQPVLTQSPSASASLGASVKLTCTLSSGHSTYAVAWHQYQPEKGPRYLMKINSDGSHIKAVGIPDRFSGSSSGAERYLTISSLQFEDEGDFYCQTWDTGIRVFGGGTKLTVLCAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 503 aa, Artificial Sequence
  Synthetic polypeptide
MEWSWVFLFFLSVTTGVHSLAEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLCAPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 19 - protein, 21 aa, Homo sapiens
MALPVTALLLPLALLLHAARP

SEQ ID NO: 20 - skipped

SEQ ID NO: 21 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - skipped

SEQ ID NO: 23 - protein, 41 aa, Artificial Sequence
  Synthetic polypeptide
RSKRSRGGHSDYMNMTPRRPGPTRKHYQPYAPPRDFAAYRS

SEQ ID NO: 24 - protein, 42 aa, Homo sapiens; identical to SEQ ID NO: 14

SEQ ID NO: 25 - DNA, 672 bp, Artificial Sequence
  Synthetic polynucleotide
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 26 - protein, 15 aa, Artificial Sequence
  Synthetic peptide
GGGGSGGGGSGGGGS

SEQ ID NO: 27 - protein, 10 aa, Artificial Sequence
  Synthetic peptide
GGGSSGGGSG

SEQ ID NO: 28 - protein, 12 aa, Artificial Sequence
  Synthetic peptide
ESKYGPPCPPCP

SEQ ID NO: 29 - protein, 12 aa, Homo sapiens
ESKYGPPCPSCP

SEQ ID NO: 30 - protein, 22 aa, Artificial Sequence
  Synthetic peptide
ESKYGPPCPPCPGGGSSGGGSG

SEQ ID NO: 31 - protein, 39 aa, Homo sapiens
IEVMYPPPYLDNEKSNGTIIHVKGKHLCPSPLFPGPSKP

SEQ ID NO: 32 - protein, 48 aa, Homo sapiens
AKPTTTPAPRPPTPAPTIASQPLSLRPEACRPAAGGAVHTRGLDFACD

SEQ ID NO: 33 - protein, 45 aa, Homo sapiens
TTTPAPRPPTPAPTIASQPLSLRPEACRPAAGGAVHTRGLDFACD

SEQ ID NO: 34 - protein, 229 aa, Artificial Sequence
  Synthetic polypeptide
ESKYGPPCPSCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 35 - protein, 229 aa, Artificial Sequence
  Synthetic polypeptide
ESKYGPPCPPCPAPEFEGGPSVFLFPPKPKDTLMISRTPEVTCVVVDVSQEDPEVQFNWYVDGVEVHQAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 36 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 37 - protein, 21 aa, Homo sapiens
LCYLLDGILFIYGVILTALFL

SEQ ID NO: 38 - protein, 27 aa, Homo sapiens
FWVLVVVGGVLACYSLLVTVAFIIFWV

SEQ ID NO: 39 - protein, 28 aa, Homo sapiens
MFWVLVVVGGVLACYSLLVTVAFIIFWV

SEQ ID NO: 40 - protein, 22 aa, Homo sapiens
MALIVLGGVAGLLLFIGLGIFF

SEQ ID NO: 41 - protein, 21 aa, Homo sapiens
IYIWAPLAGTCGVLLLSLVIT

SEQ ID NO: 42 - protein, 23 aa, Homo sapiens
IYIWAPLAGTCGVLLLSLVITLY

SEQ ID NO: 43 - protein, 24 aa, Homo sapiens
IYIWAPLAGTCGVLLLSLVITLYC

SEQ ID NO: 44 - protein, 27 aa, Homo sapiens
IISFFLALTSTALLFLLFFLTLRFSVV

SEQ ID NO: 45 - protein, 41 aa, Homo sapiens
RSKRSRLLHSDYMNMTPRRPGPTRKHYQPYAPPRDFAAYRS

SEQ ID NO: 46 - protein, 42 aa, Homo sapiens
ALYLLRRDQRLPPDAHKPPGGGSFRTPIQEEQADAHSTLAKI